(19) United States
(12) Patent Application Publication
(10) Pub. No.: US 2025/0009842 A1
(43) Pub. Date: Jan. 9, 2025

(54) Methods for Regulating Muscle Performance using Fat Specific Protein 27 (FSP27) Compositions

(71) Applicant: Ohio University, Athens, OH (US)

(72) Inventors: Vishwajeet PURI, Athens, OH (US); Bijinu BALAKRISHNAN, Athens, OH (US)

(73) Assignee: Ohio University, Athens, OH (US)

(21) Appl. No.: 18/802,702

(22) Filed: Aug. 13, 2024

Related U.S. Application Data
(63) Continuation-in-part of application No. PCT/US2023/062024, filed on Feb. 6, 2023.
(63) Continuation-in-part of application No. 17/873,386, filed on Jul. 26, 2022, now Pat. No. 11,925,674.
Application No. 17/873,386 is a division of application No. 16/620,661, filed on Dec. 9, 2019, now Pat. No. 11,433,117.
(60) Provisional application No. 63/309,881, filed on Feb. 14, 2022.
(60) Provisional application No. 62/520,015, filed on Jun. 15, 2017.

(51) Int. Cl. A61K 38/17; A61K 9/00; A61P 3/00; A61P 3/06; A61P 3/10

(52) U.S. Cl. CPC A61K 38/1709; A61P 3/00; A61P 3/06; A61P 3/10; A61K 9/0019

(57) ABSTRACT

Methods and FSP27 compositions for increasing muscle performance and/or regulating lipid droplet dynamics to store triglycerides in the muscles, and regulating their breakdown via lipolysis are described.

### STATEMENT REGARDING FEDERALLY SPONSORED RESEARCH

This invention was made with government support under RO1DK101711 and RO1HL140836 awarded by National Institutes of Health. The government has certain rights in the invention.

### CROSS-REFERENCE TO RELATED APPLICATIONS

This application is a continuation in part of international application PCT/US2023/062024, filed under the authority of the Patent Cooperation Treaty on Feb. 6, 2023, which claims priority to U.S. Provisional Application Ser. No. 63/309,881 filed Feb. 14, 2022, this application is a continuation-in-part of Ser. No. 17/873,386 filed Jul. 26, 2022, now US. Pat. No. 11,925,674 issued Mar. 12, 2024, which is a divisional application of Ser. No. 16/620,661 filed Dec. 9, 2019, now US Pat. No. 11,433, 117 issued Sep. 6, 2022, which is a 371 application of PCT/US2018/037443 filed Jun. 14, 2018, which claims the priority to U.S. Provisional Application Ser. No. 62/520,015 filed Jun. 15, 2017, the entire disclosures of which are expressly incorporated herein by reference.

### Sequence Listing

The instant application contains a Sequence Listing which has been submitted electronically in XML format and is hereby incorporated by reference in its entirety. Said XML copy, created on Sep. 4, 2024, is named 2024-10-02_SEQList_63311-US-CIP_OU22006-US_SL.xml and is 8,850 bytes in size.

### BACKGROUND OF THE INVENTION

It is well established that there is impaired fat storage and breakdown, which leads to increased circulatory free fatty acids (FFAs; also referred to as unesterified fatty acids). High levels of circulating FFAs are a major risk factor for the lipotoxicity. Ectopic deposition of these FFAs impair insulin signaling in various tissues and organs like liver, muscle and pancreas with the onset of insulin resistance in patients with type 2 diabetes and/or obesity.

Fat specific protein 27 (FSP27) also known as CIDEC (Cell Death Inducing DFFA like Effector C) has recently been identified as being associated with lipid droplets in adipocytes. It is an intracellular protein that has also been shown to be expressed in muscle and liver tissue. FSP27 is strikingly up-regulated during adipogenesis and is highly expressed in adipose tissue. FSP27 depletion in adipocytes causes increased lipolysis, resulting in breakdown of triglycerides into free fatty acids. FSP27 expression in omental fat positively correlates with insulin sensitivity in obese humans. Also, a homozygous nonsense mutation, FSP27 E186X, in a human subject has been shown to be associated with a phenotype of partial lipodystrophy and insulin resistant diabetes.

The nonsense mutation (E186X) in Fsp27 causes increased lipolysis, lipodystrophy, and insulin-resistant type 2 diabetes in humans, highlighting a positive association of Fsp27 with improved metabolic health. However, mouse models of Fsp27 have unveiled confusing results. Whole-body Fsp27^{-/-} mice have reduced levels of circulatory triglycerides (TGs) and show smaller lipid droplets in their white fat adipocytes. However, Fsp27^{-/-} mice were protected from diet-induced obesity (DIO), resulting in improved glucose disposal and insulin sensitivity. Furthermore, Fsp27-deficiency led to improved metabolic rates by upregulating genes involved in mitochondrial oxidative metabolism and browning of white adipose tissue. Similarly, silencing of Fsp27 using intraperitoneal injection of antisense oligonucleotides in high-fat diet (HFD) fed mice and ob/ob mice resulted in decreased visceral adiposity, improved insulin sensitivity, and whole-body glycemic control. In contrast, adipose-specific Fsp27^{-/-} mice were resistant to HFD-induced weight gain, but impaired lipid storage in adipose tissue resulted in hepatosteatosis and systemic insulin resistance.

There is no admission that the background art disclosed in this section legally constitutes prior art.

### SUMMARY OF THE INVENTION

In a first broad aspect, described herein are uses of FSP27 compositions. It is now described herein that the exogenous delivery of FSP27 peptides are able to rescue FSP27 dysfunction.

In another broad aspect, described herein is a method for improving muscle performance in a subject in need thereof, by administering to the subject an FSP27 medicament or a pharmaceutically acceptable composition thereof.

In another broad aspect, described herein is a method for regulating lipid droplet dynamics as a way to store triglycerides in the muscles, and regulating lipid droplet breakdown via lipolysis in a subject in need thereof by administering to the subject an FSP27 medicament or a pharmaceutically acceptable composition thereof.

In certain embodiments, the FSP27 medicament is administered to muscle tissue.

In certain embodiments, muscle performance includes higher endurance and less fatigability, than is a subject not receiving the FSP27 medicament.

Also described herein are methods of treatment where administering exogenous recombinant FSP27 (rFSP27) as a therapeutic improves insulin signaling in adipocytes and adipose tissue of humans, resulting in decreased insulin resistance.

Such uses include, but are not limited to, increasing levels of FSP27 in a subject by administering exogenous recombinant FSP27 (rFSP27), where the subject is suffering from a metabolic disease and/or other diseases associated with increased free fatty acids and/or lipotoxicity, and conditions associated with these diseases.

In certain embodiments, the metabolic disease and conditions associated with the disease are one or more of insulin resistance, obesity, and dyslipidemia.

In certain embodiments, the other diseases associated with increased free fatty acids and/or lipotoxicity and conditions associated with these diseases are one or more of Type 2 diabetes, fatty liver disease, hypothyroidism, gout, hernia, Pickwickian syndrome, lymph edema, cellulitis, depression, polycystic ovary syndrome, urinary incontinence, chronic renal failure, and erectile dysfunction.

Another use of FSP27 compositions is for reducing visceral obesity, insulin resistance and improving blood glucose levels by administering an effective amount of rFSP27 to improve insulin induced signaling in cells or whole body in a subject in need thereof.

Another use of FSP27 compositions is for modulating lipolysis in adipocytes, by administering rFSP27 to a subject in an amount sufficient to protect that subject from insulin resistance.

Another use of FSP27 compositions is for regulating lipid droplet morphology and optimizing storage and breakdown of fat (lipolysis) in a subject, by administering an effective amount of rFSP27 to a subject in need thereof.

Another use of FSP27 compositions is method for decreasing adipose tissue glycerol lipase (ATGL) expression and lipolysis in a subject in need thereof, by administering an effective amount of rFSP27.

Another use of FSP27 compositions is for protecting adipocyte cells against FFA-induced insulin resistance, by administering an effective amount of rFSP27 to a subject in need thereof.

Another use of FSP27 compositions is for treating pathologies associated with insulin-resistance syndrome, by the administration of an efficient amount of a FSP27 composition to a subject in need thereof. Such pathologies can include, for example, for the treatment of Type 2 diabetes in a subject.

In another broad aspect, described herein are pharmaceutical compositions comprising one or more FSP27 medicaments. FSP27 medicaments may be administered as a pharmaceutically acceptable salt, or as a pegylated composition, or be modified in a pharmaceutically acceptable manner so as to improve the therapeutic properties. FSP27 medicaments may also be administered optionally together with one or more inert carriers and/or diluents.

In certain embodiments, the subject is a human.

In certain embodiments, the nucleic acid encoding the FSP27 protein is operably linked to a constitutive promoter/enhancer, an adipocyte-specific promoter/enhancer, or an inducible promoter/enhancer.

In certain embodiments, the composition comprises a plasmid, the plasmid comprising the nucleic acid encoding the FSP27 protein operably linked to a transcriptional regulatory sequence (promoter/enhancer).

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIGS. 1A-1D: Schematic illustration of a model of FSP27 regulation of lipolysis. A model of FSP27 regulation of lipolysis that is supported by the results described herein. PLIN1 scaffolds FSP27 at the lipid droplet surface where FSP27 interacts with ATGL and decreases lipolysis:

FIG. 1A. In basal conditions, FSP27 decreases the access of ATGL to its coactivator CGI-58, thereby diminishing lipolysis, as indicated by the dashed downward arrow.

FIG. 1B. When FSP27 is absent in basal conditions, ATGL is free to interact with CGI-58, leading to increased lipolysis, as indicated by solid downward arrow.

FIG. 1C. Upon B-adrenergic stimulation in the presence of FSP27, PKA activation results in phosphorylation of PLIN1 and HSL, causing release of CGI-58 which binds to and stimulates ATGL. Unbound ATGL is translocated to lipid droplet and G0S2 is downregulated to increase ATGL-mediated lipolysis as indicated by the bolded downward arrow.

FIG. 1D. Upon B-adrenergic stimulation in the absence of FSP27, the otherwise FSP27-sequestered-ATGL is now available for CGI-58 binding, resulting in even higher levels of lipolysis, indicated by the more prominent downward arrow.

FIGS. 2A-2C: FSP27 depletion increased both basal and stimulated lipolysis in human adipocytes:

FIG. 2A. Relative mRNA levels in siRNA-transfected human adipocytes.

FIG. 2B. Immunoblot and quantification of protein expression levels of FSP27 and β-tubulin (loading control) of siRNA-transfected human adipocytes.

FIG. 2C. Biochemical quantification of basal and stimulated lipolysis based on

measurement of glycerol release after 2 hours.

FIG. 3: FSP27 expression decreased ATGL-mediated lipolysis.

FIGS. 4A-4B: FSP27 negatively regulates ATGL expression and lipolysis in human adipocytes:

FIG. 4A. RNA was extracted from control and siRNA-treated adipocytes, and mRNA levels were measured by quantiative PCR and normalized by GAPDH mRNA.

FIG. 4B. Protein lysates from control and siRNA-treated adipocytes were loaded at 15 μg/lane and probed with antibodies against FSP27, ATGL or B-tubulin.

FIGS. 5A-5G: FSP27 inhibits ATGL promoter activity via Egr1:

FIGS. 5A, 5B, 5D, 5F, and 5G. HEK293T cells cultured in 12-well dishes were transfected with the full length (−2979/+21), C→T mutated, or truncated ATGL luciferase promoter constructs, cDNA for eGFP; cDNAs for FSP27 and Egr1 as well as scrambled siRNA and Egr1 siRNA as indicated.

FIG. 5C. Schematic representation of the proximal region of ATGL promoter with the consensus Egr1 binding site. [SEQ ID NO:1].

FIG. 5D. Indicates the synergistic effect between Egr1 and FSP27 with p<0.05.

FIG. 5E. HEK293T cells growing in 35 mm dishes were transfected with scrambled or Egr1 siRNA.

FIGS. 6A-6D: FSP27 protected human adipocytes against FFA-induced insulin resistance:

FIG. 6A: Insulin stimulated AKT phosphorylation in human adipocytes after siRNA-mediated FSP27 knockdown.

FIG. 6B. Insulin stimulated AKT phosphorylation in human adipocytes in the presence or absence of FSP27-CFP or EGFP (Control).

FIG. 6C. FSP27-HA expression protects adipocytes.

FIG. 6D. 100 μM PA/BSA or FSP27-HA expression had no effect on insulin stimulated AKT activation in adipocytes differentiated from ATGL-KO MEFs.

FIG. 7: Basal lipolysis was significantly higher in visceral depots compared to subcutaneous. Glycerol release was measured in 12 subcutaneous, 15 omentum adipose depots and normalized to total ug of protein. Data are presented as ±SEM.

FIGS. 8A-8B: Increased lipolysis in visceral adipose negatively correlates with FSP27 expression:

FIG. 8A. Basal FSP27 was significantly higher in subcutaneous depot.

FIG. 8B. Basal FSP27 protein was measured in 13 paired subcutaneous, and omentum depots.

FIGS. 9A-9B: siRNA-mediated FSP27 knockdown increases lipolysis and impairs insulin signaling:

FIG. 9A. Knockdown of FSP27 in subcutaneous adipose tissue increased rate of glycerol release in the media.

FIG. 9B. siRNA-mediated FSP27 depletion decreased Akt phosphorylation.

FIGS. 10A-10B: Recombinant FSP27 improves insulin signaling in visceral adipose:

FIG. 10A. Treatment of visceral depot with recombinant FSP27 decreased basal lipolysis.

FIG. 10B. Quantification of insulin-stimulated AKT phosphorylation.

FIG. 11: FSP27 (120-220) protected against FFA-induced insulin resistance in human primary adipocytes.

FIG. 12: Schematic representation of FSP27 and its functional domains: CIDE-N and CIDE-C.

FIG. 13: FSP27 sequence is conserved in vertebrates; for example, >90% conserved sequence in FSP27 in humans, mouse, monkey, dog, cow and frog. FIG. 13 discloses SEQ ID NOs: 2-7, respectively, in order of appearance.

FIG. 14A: Insulin tolerance test (ITT) in AT-hFSP27tg mice.

FIG. 14B: Glucose tolerance test (GTT) in AT-hFSP27tg mice.

FIG. 15A: Glucose tolerance test (GTT) in FSP27^{-/-} mice.

FIG. 15B: Insulin tolerance test (ITT) in FSP27^{-/-} mice.

FIG. 16A: Fasting blood insulin in FSP27 knockout mice.

FIG. 16B: Non-esterified fatty acid (NEFA) (Free fatty acids) in FSP27 knockout mice.

FIGS. 17A-17F: Weight, food, and water consumption of mice. Body-weight (FIG. 17A; in grams) and body-fat percentage (FIG. 17B) of wild type, Fsp27^{+/-} (Het-KO) and Fsp27^{-/-} (Fsp27-KO) mice. Food consumption over 96 hours (FIG. 17C) for each mouse genotype in male mice. The right-hand side panel shows the quantification of total food consumption in four days. Tissue mass was weighed immediately after dissection in male (FIG. 17D) and female (FIG. 17E) mice. Water consumption over 96 hours (17F) for each mouse genotype. The right-hand side panel shows quantification of total water consumption in four days (n=6 mice for each group). Data are expressed as means ±S.E.M. For statistical analysis, one-way ANOVA followed by Tukey's multiple comparison test was performed, *p<0.05, ** p<0.01, *** p<0.001, **** p<0.0001.

FIGS. 18A-181: Glucose and insulin tolerance testing in mice. Data are graphed over time for male and female mice. Glucose Insulin tolerance test (GTT) of males (FIG. 18A). The area under the curve (AUC) of GTT for males (FIG. 18B). Insulin tolerance test (ITT) of males (FIG. 18C). AUC of ITT for males (FIG. 18D). GTT of females (FIG. 18E). AUC of GTT for females (FIG. 18F). ITT of females (FIG. 18G). AUC of ITT for females (FIG. 18H). Fasted basal glucose, taken after overnight fasting, for both male and female mice (FIG. 181). WT, wild type; Het-KO, Fsp27^{+/-} mouse; Fsp27-KO, Fsp27^{-/-} mouse (n=6 mice for each group). Data are expressed as means ±S.E.M. For statistics on FIG. 18A, FIG. 18C, FIG. 18E, and FIG. 18G, two-way ANOVA followed by Bonferroni posthoc analysis was performed; for statistics on FIG. 18B, FIG. 18D, FIG. 18F, and FIG. 18H, one-way ANOVA followed by Tukey's multiple comparison test was performed, *p<0.05, ** p<0.01, *** p<0.001.

FIGS. 19A-19D: Metabolic phenotyping of each mouse genotype. Mice were monitored through metabolic chambering for a total of 5 days. Measurements were taken during the day cycle (6:00 AM to 6:00 PM) and the night cycle (6:00 PM to 6:00 AM) were averaged within each respective time cycle. The respiratory exchange ratio for day and night cycles (FIG. 19A). VO2 19 (FIG. 19B). VCO2 (FIG. 19C). Heat generation through indirect calorimetry (FIG. 19D). Het-KO, Fsp27^{-/-} mouse; Fsp27-KO, Fsp27^{-/-} mouse (n>6 male mice per group). Data are expressed as means +S.E.M. For statistics, one-way ANOVA followed by Tukey's multiple comparison tests was performed, *p<0.05, ** p<0.01, *** p<0.001, **** p<0.0001.

FIGS. 20A-20F: Locomotor activity of each mouse genotype. Mice were monitored through metabolic chambering for a total of 5 days. Measurements were taken during the day cycle (6:00 AM to 6:00 PM) and the night cycle (6:00 PM to 6:00 AM) were averaged within each respective time cycle. Mouse movement along the X-axis, either ambulatory (FIG. 20A, FIG. 20B). Total X-axis movement (FIG. 20C, FIG. 20D). Z-axis movement (FIG. 20E, FIG. 20F). Het-KO, Fsp27^{-/-} mouse; Fsp27-KO, Fsp27^{-/-} mouse (n≥6 male mice per group). Data are expressed as means ±S.E.M. n≥6 male mice per group. For statistics, one-way ANOVA followed by Tukey's multiple comparison tests was performed separately for day and night cycle, *p<0.05, ** p<0.01, *** p<0.001.

FIGS. 21A-21E: Muscle physiology testing and fiber typing. Treadmill running endurance was measured with an Exer 3/6 Metabolic Treadmill (FIG. 21A). After acclimation, mice (n>10 mice per group) were allowed to run until exhaustion as defined by spending over 5 consecutive seconds in the fatigue zone (see Methods). Results from the four-limb hanging grid test performed on mice (n>10 mice per group) with a minimum of 5 trials (FIG. 21B). Total hang time was used to calculate holding impulse after converting the weight of the mouse into Newtons. Fiber typing of the gastrocnemius (FIG. 21C), tibialis anterior (FIG. 21D) and extensor digitorum longus (FIG. 21E) muscle sections with multiplex immunofluorescent antibody staining (n=3 male mice per group). WT, wild type; Het-KO, Fsp27^{-/-} mouse; Fsp27-KO, Fsp27^{-/-} mouse. Data are expressed as means ±S.E.M. For statistics, one-way ANOVA followed by Tukey's multiple comparison tests was performed separately for each fiber types, *p<0.05, ** p<0.01, *** p<0.001.

FIGS. 22A-22G: Lipid and glycogen analysis. Serum collected from fasted wild type and Fsp27-/mice (n=5) was analyzed for total free fatty acids (FFA) and triglycerides (TG; FIG. 22A) as well as the lipid species composition (FIG. 22B-FIG. 22E). For (FIG. 22A) one-way ANOVA followed by Bonferroni posthoc analysis was performed. Data for the amount of each lipid species in the male TG fraction (FIG. 22B). Lipid species in female TG fraction (FIG. 22C). Lipid species in male FFA fraction (FIG. 22D). Lipid species in female FFA fraction (FIG. 22E). For (FIG. 22B-FIG. 22E) two-way ANOVA followed by Bonferroni posthoc analysis was performed. Liver (FIG. 22F) and muscle (FIG. 22G) glycogen content in fasted and unfasted wild type and Fsp27^{-/-} male mice (n=3 mice per group). WT, wild type; Fsp27-KO, Fsp27^{-/-} mouse. All data are expressed as means ±S.E.M. For statistics (F&G) one-way ANOVA followed by Tukey's multiple comparison test was performed, *p<0.05, ** p<0.01, *** p<0.001, **** p<0.0001.

FIGS. 23A-231: Energy reserves, fuel utilization, muscle mass, and mitochondrial/hepatic output during exercise in WT and FSP27^{-/-} mice. Soleus muscle mass (FIG. 23A), Gastrocnemius muscle mass (FIG. 23B). q-PCR analysis of mitochondrial genes normalized to β-actin (FIG. 23C). Western blot analysis of mitochondrial proteins: cytochrome C, COX IV, and Voltage Dependent Anion Selective Channel 1 (FIG. 23D). Serum glucose levels (FIG. 23E), FFA levels (FIG. 23F), Liver (FIG. 23G), and muscle (FIG. 23H) glycogen levels. Blood lactate levels before and after exercise in WT and FSP27^{-/-} animals (FIG. 231). WT, wild type; FSP27^{-/-} male mice (n=5 male mice per group). Data are expressed as means ±S.E.M. For statistics, unpaired t test was performed for FIG. 23A-FIG. 23B, two-way ANOVA followed by Tukey's multiple comparison test was performed for FIG. 23E-FIG. 231. For C two-Way ANOVA followed by Šídák's multiple comparisons test was used to analyze the significance between the groups * p<0.05, ** p<0.01 *** p<0.001, ****p<0.0001.

FIGS. 24A-24C: Localization of fat and Fsp27 in muscle. (FIG. 24A) Confocal microscopy images representing the localization of Fsp27 (magenta; Cy5), neutral lipid (green; BODIPY), and nuclei (blue; DAPI) in the gastrocnemius of wild type, Fsp27^{+/-} and Fsp27^{-/-} mice. (FIG. 24B) Qualitative measurement of fat staining in the gastrocnemius of wild type, Fsp27^{+/-} and Fsp27^{-/-} mice. (FIG. 24C) Intramuscular triglyceride contents. n=5 male mice per group. Data are expressed as means ±S.E.M. For statistics one-way ANOVA followed by Tukey's multiple comparison test was performed * p<0.05, ** p<0.01 *** p<0.001, **** p<0.0001.

FIGS. 25A-25C: FSP27^{-/-} mice showed reduces muscle performance in at 2-3 months of age. Four-limb hanging grid test performed on female mice (FIG. 25A), male mice (FIG. 25B). For treadmill running endurance test mice was run until exhaustion Male mice (FIG. 25C) female mice (FIG. 25D) (n≥8 mice per group).

FIGS. 26A-26D: FSP27^{-/-} mice showed reduces muscle performance in at 5-6 months of age. Four-limb hanging grid test performed on female mice (FIG. 26A), male mice (FIG. 26B). For treadmill running endurance test mice was run until exhaustion female mice (FIG. 26C) male mice (D) (n>8 mice per group).

### DETAILED DESCRIPTION OF THE PREFERRED EMBODIMENT(S)

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

### Definitions

In order to facilitate review of the various embodiments of the disclosure, the following explanations of specific terms are provided:

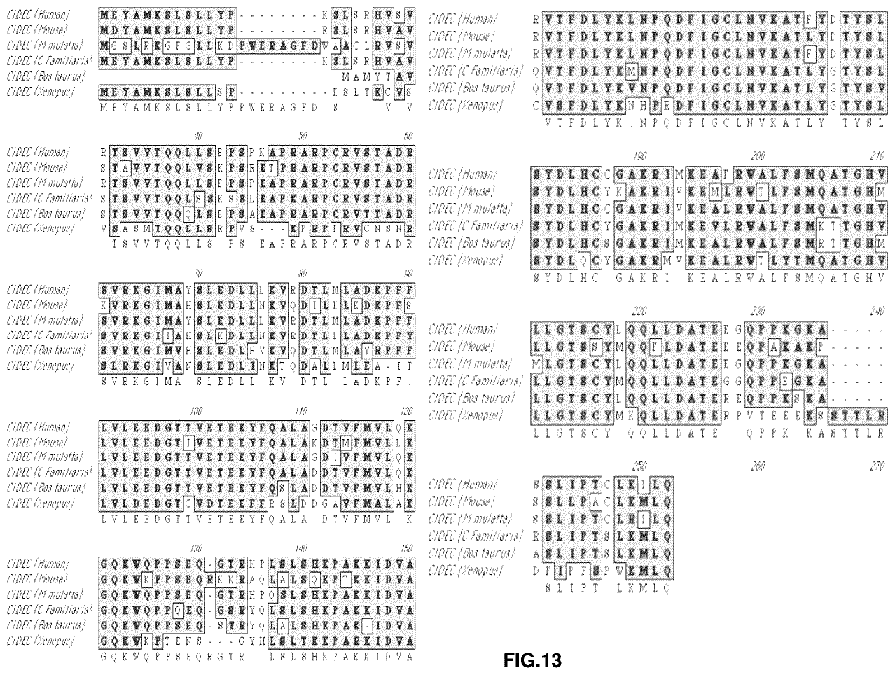

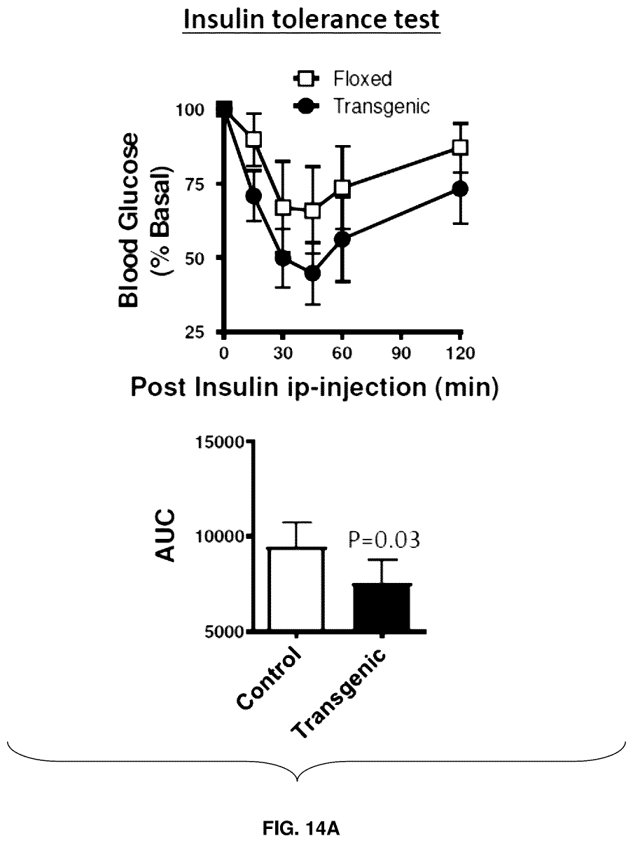

FSP27 Compositions/Medicaments: Refers to the FSP27 as shown in the schematic representation of FSP27 and its functional domains in FIG. 12, including any substitutions, deletions, modifications, or mutations thereof. FSP27 Compositions/Medicaments as contemplated herein may also be prepared as recombinant proteins, including the FSP27 sequences shown in FIG. 13.

The FSP27 protein may also be encoded by nucleic acids. As used herein, a “nucleic acid” or “polynucleotide” includes a nucleic acid, an oligonucleotide, a nucleotide, a polynucleotide, and any fragment or variant thereof. The nucleic acid or polynucleotide may be double-stranded, single-stranded, or triple-stranded DNA or RNA (including cDNA), or a DNA-RNA hybrid of genetic or synthetic origin, wherein the nucleic acid contains any combination of deoxyribonucleotides and ribonucleotides and any combination of bases, including, but not limited to, adenine, thymine, cytosine, guanine, uracil, inosine, and xanthine hypoxanthine. The nucleic acid or polynucleotide may be combined with a carbohydrate, lipid, protein, or other materials. Preferably, the nucleic acid encodes FSP27 protein.

The “complement” of a nucleic acid refers, herein, to a nucleic acid molecule which is completely complementary to another nucleic acid, or which will hybridize to the other nucleic acid under conditions of high stringency. High-stringency conditions are known in the art (see e.g., Maniatis et al., Molecular Cloning: A Laboratory Manual, 2nd ed. (Cold Spring Harbor: Cold Spring Harbor Laboratory, 1989) and Ausubel et al., eds., Current Protocols in Molecular Biology (New York, N.Y.: John Wiley & Sons, Inc., 2001)). Stringent conditions are sequence-dependent, and may vary depending upon the circumstances. As used herein, the term “cDNA” refers to an isolated DNA polynucleotide or nucleic acid molecule, or any fragment, derivative, or complement thereof. It may be double-stranded, single-stranded, it may have originated recombinantly or synthetically, and it may represent coding and/or noncoding 5′ and/or 3′ sequences.

In addition, “complementary” means not only those that are completely complementary to a region of at least 20 continuous nucleotides, but also those that have a nucleotide sequence homology of at least 40% in certain instances, 50% in certain instances, 60% in certain instances, 70% in certain instances, at least 80%, 90%, and 95% or higher. The degree of homology between nucleotide sequences can be determined by an algorithm, BLAST, etc.

As used herein nucleic acids and/or nucleic acid sequences are “homologous” when they are derived, naturally or artificially, from a common ancestral nucleic acid or nucleic acid sequence. Homology is generally inferred from sequence identity between two or more nucleic acids or proteins (or sequences thereof). The precise percentage of identity between sequences that is useful in establishing homology varies with the nucleic acid and protein at issue. Higher levels of sequence identity, e.g., 30%, 40%, 50%, 60%, 70%, 80%, 90%, 95%, 96%, 97%, 98%, or 99% or more can also be used to establish homology. Methods for determining sequence similarity percentages (e.g., BLASTN using default parameters) are generally available. Software for performing BLAST analyses is publicly available through the National Center for Biotechnology Information.

The nucleic acid agent, for example, may be a plasmid. Such a plasmid may comprise a nucleic acid sequence encoding FSP27 or another FSP27-associated protein, although it is to be understood that other types of nucleic acid agents, such as recombinant viral vectors, may also be used for the purposes of the present invention. In one embodiment of the present invention, the nucleic acid (e.g., plasmid) encodes at least one FSP27-associated protein.

The term “plasmid”, as used herein, refers generally to circular double-stranded DNA, which is not bound to a chromosome. The DNA, for example, may be a chromosomal or episomal-derived plasmid. The plasmid of the present invention may optionally contain a promoter/enhancer and terminator of transcription, and/or a discrete series of restriction-endonuclease recognition sites, located between the promoter and the terminator. In the plasmid, a polynucleotide insert of interest (e.g., one encoding a FSP27-associated protein) should be operatively linked to an appropriate promoter. The promoter may be its native promoter or a host-derived promoter. The promoter may also be a tissue-specific promoter, such as an adipocyte-specific promoter or other tissue-specific promoter. The promoter may further be a regulatable promoter, which may be turned off when the expression of the gene is no longer desired. Non-limiting examples of promoters for use in the present invention include the actin or albumin promoter and viral promoters. Other suitable promoters will be known to the skilled artisan.

Therapeutic: A generic term that includes both diagnosis and treatment. It will be appreciated that in these methods the “therapy” may be any therapy for treating a disease including, but not limited to, pharmaceutical compositions, gene therapy and biologic therapy such as the administering of antibodies and chemokines. Thus, the methods described herein may be used to evaluate a patient or subject before, during and after therapy, for example, to evaluate the reduction in disease state.

Adjunctive therapy: A treatment used in combination with a primary treatment to improve the effects of the primary treatment.

Clinical outcome: Refers to the health status of a patient following treatment for a disease or disorder or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

Decrease in survival: As used herein, “decrease in survival” refers to a decrease in the length of time before death of a patient, or an increase in the risk of death for the patient.

Patient: As used herein, the term “patient” includes human and non-human animals. The preferred patient for treatment is a human. “Patient,” “individual” and “subject” are used interchangeably herein.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop or stops the progression of said disease. “Ameliorating” refers to the reduction in the number or severity of signs or symptoms of a disease.

Poor prognosis: Generally refers to a decrease in survival, or in other words, an increase in risk of death or a decrease in the time until death. Poor prognosis can also refer to an increase in severity of the disease, such as an increase in spread (metastasis) of the cancer to other tissues and/or organs.

Screening: As used herein, “screening” refers to the process used to evaluate and identify candidate agents that affect such disease.

Comprising, comprises and comprised of: As used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

About: As used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

And/or: When used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

Metabolic diseases: As used herein means any disease caused by an abnormal metabolic process that may be congenital, resulting from an inherited abnormality, or acquired, resulting from organ or system dysfunction or failure.

### EXAMPLE 1:

### General Description

Lipotoxicity due to excess adipose tissue lipolysis contributes to insulin-resistance. Fat

Specific Protein (FSP27) is a key regulator of lipolysis in adipocytes. Lipotoxicity can exist in muscle, liver, pancreas and other organs. FIG. 12 provides a schematic representation of FSP27 and its functional domains.

FSP27 is a lipid droplet associated protein that regulates fatty acid homeostasis in adipocytes, and its expression is inversely associated with insulin sensitivity in obese humans. Human genetic FSP27 mutations are associated with lipodystrophy, hypertriglyceridemia, and insulin-resistance and inactivating mutations of FSP27 in humans leads to increased lipolysis. In addition, adipose-specific disruption of FSP27 causes insulin resistance in high fat fed mice.

### Lipolysis in the Pathogenesis of Insulin Resistance.

Lipolysis is a catabolic branch of the fatty acid (FA) cycle that provides FAs in times of metabolic need. FAs are essential substrates for energy production and the synthesis of most lipids. Despite their fundamental physiological importance, an oversupply of FAs is highly detrimental. Increased free fatty acids (FFAs) cause lipotoxicity which disrupt the integrity of membranes, alters cellular acid-base homeostasis, and elicits the generation of harmful bioactive lipids. These effects, in turn, impair membrane function and induce endoplasmic reticulum (ER) stress, mitochondrial dysfunction, inflammation, cell death, and insulin resistance. Adipose tissue regulates the balance of FA esterification and triglyceride (TG) lipolysis, thus playing a central role in regulating whole body metabolism and glucose homeostasis. High concentrations of circulating FFAs and TG, observed in both obesity and lipodystrophy, are believed to cause insulin resistance and decreased glucose tolerance.

### FSP27 Regulates Lipid Droplet Morphology and Lipolysis:

The FSP27 protein associates with lipid droplets and regulates FA homeostasis in adipocytes. FSP27 regulates lipid droplet dynamics and lipolysis in adipocytes through regulation of the catalytic capacity as well as transcription of adipose tissue glycerol lipase, ATGL, the rate-limiting enzyme in lipolysis. FSP27 levels are inversely associated with insulin sensitivity in obese humans, and mutation of FSP27 in humans leads to increased lipolysis. In addition, adipose-specific disruption of FSP27 causes insulin resistance in high fat fed mice.

Described herein is a previously unrecognized role of FSP27 in the regulation of insulin signaling to protect against insulin resistance and Type 2 diabetes. While metabolic regulation of FSP27 has been essentially characterized exclusively in adipocytes, the data herein show that FPS27 is down-regulated particularly in association with visceral/central obesity. Perturbations in FSP27 may promote conditions that elevate FFAs, which cause or promote insulin resistance. However, lipid storage/breakdown is generally not viewed as a primary function of other cell types, thus FSP27 may govern cellular responses by mechanisms beyond regulation of lipid metabolism.

Moreover, at the local adipose tissue level, capillary rarefaction and impaired perfusion have been linked to adipose tissue pseudohypoxia and metabolic dysregulation. FSP27 is down-regulated in human visceral fat and is associated with insulin resistance.

Also described herein are mouse models that are adipose specific transgenic mice expressing human FSP27. These mice are useful to examine the relative contribution of adipose in regulating insulin resistance and/or Type 2 diabetes.

### FS-IVGTT

Fasting subjects are studied in the morning in the general clinical research center (GCRC). Any oral diabetic medication is held 48 hrs prior to testing. Two intravenous forearm catheters are placed in each arm (one for sampling and one for infusion). Baseline blood samples are collected at t=−15 min and t=−5 min) for measurement of glucose and insulin. A bolus of 300 mg/kg glucose in 25% glucose/saline infusion over 1 minute is given. At t=20 min, a bolus of 0.05 units/kg of regular insulin intravenously which improves accuracy of the FS-IVGTT in diabetic subjects is given. Blood is collected for insulin and glucose at t=2, 3, 4, 5, 6, 8, 10, 14, 16, 19, 22, 25, 30, 40, 50, 60, 70, 90, 110, 130, 150, 170, and 180 minutes. The insulin sensitivity index (SI) and disposition index (DI) are calculated using the MINMOD software based on the Bergman model.

These data collected within 1 week of the planned surgery.

### Quantitative Real-Time PCR and Western Blot Analyses

Using Quantitative real-time PCR, fat tissue expression of specific mediators relevant to insulin signaling, FSP27, and inflammation (which will initially consist of: TNFα, IL-6, IL-1β, MCP-1, CD68, FSP27, IRS-1, PI3-K, Akt, PTEN) are examined.

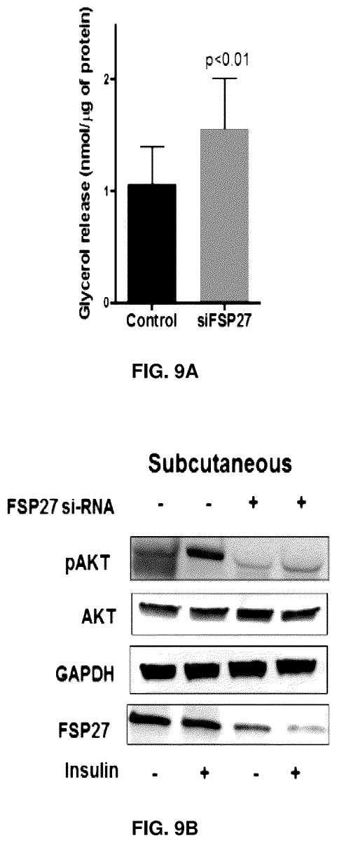

FSP27 is associated with lipid droplets and functions primarily as a regulator of lipid droplet morphology and lipolysis in adipocytes. Visceral adipose tissue showed lower FSP27 expression as compared to subcutaneous depots (see FIGS. 9A-9B).

### FSP27 Prevents the Interaction of ATGL with its Aactivator, CGI-58, Leading to a Decreased Lipolysis in Human Adipocytes.

Lipolysis of TGs to FAs and glycerol requires three consecutive steps that involve three different enzymes, Adipose tissue glycerol lipase (ATGL; also called desnutrin and PNPLA2), Hormone sensitive lipase (HSL), and Monoacylglycerol lipase (MGL). ATGL is the rate-limiting enzyme for lipolysis in adipocytes which catalyzes the first step of hydrolysis of TG to diacyl glycerol (DG).

### RESULTS of Example 1:

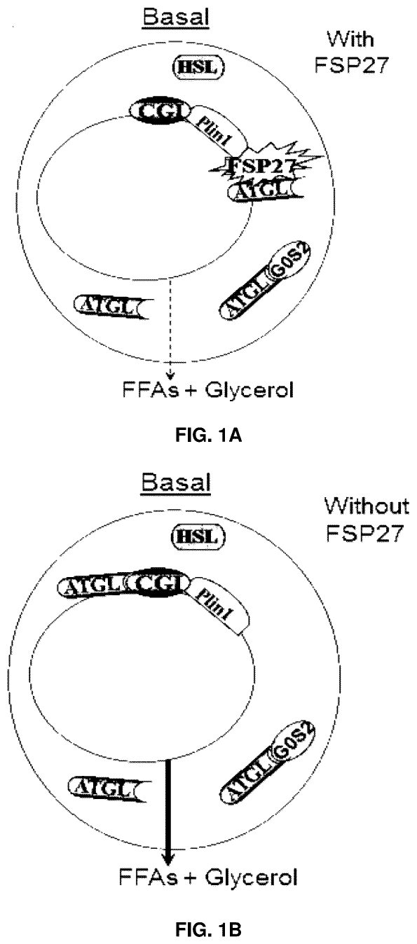

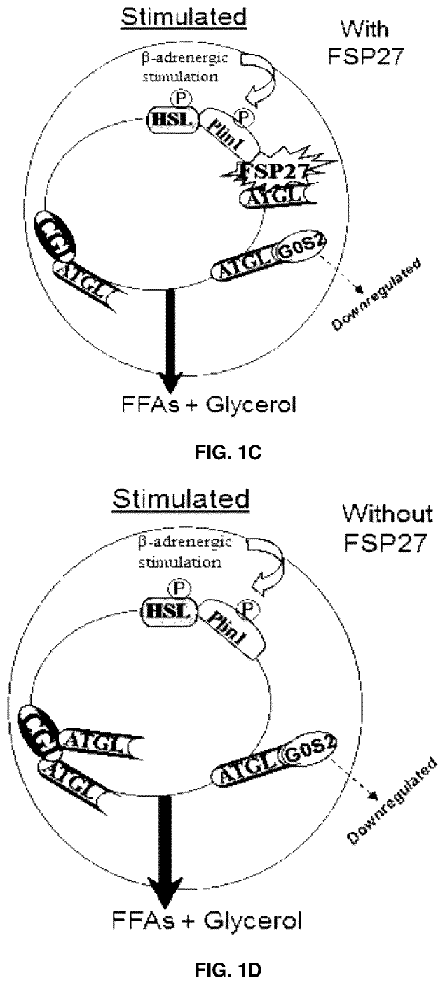

FSP27 regulates lipolysis in adipocytes through regulation of the catalytic capacity as well as transcription of the adipose tissue glycerol lipase (ATGL) gene. The activity of ATGL depends upon its interaction with its activator CGI-58. As shown in FIGS. 1A-1D, FSP27 interacts with ATGL at lipid droplet surface and inhibits its interaction with the activator CGI-58, thus preventing the activation of ATGL to regulate lipolysis under both basal and stimulated conditions.

FIGS. 1A-1D provide a schematic illustration of a model of FSP27 regulation of lipolysis. FIG. 1A illustrates that, in basal conditions, FSP27 decreases the access of ATGL to its coactivator CGI-58, thereby diminishing lipolysis, as indicated by the dashed downward arrow.

FIG. 1B illustrates that, when FSP27 is absent in basal conditions, ATGL is free to interact with CGI-58, leading to increased lipolysis, as indicated by solid downward arrow.

FIG. 1C illustrates that, upon β-adrenergic stimulation in the presence of FSP27, PKA

activation results in phosphorylation of PLIN1 and HSL, causing release of CGI-58, which binds to and stimulates ATGL. Unbound ATGL is translocated to lipid droplet and G0S2 is downregulated to increase ATGL-mediated lipolysis as indicated by the bolded downward arrow.

FIG. 1D illustrates that, upon β-adrenergic stimulation in the absence of FSP27, the otherwise FSP27-sequestered-ATGL is now available for CGI-58 binding, resulting in even higher levels of lipolysis, indicated by the more prominent downward arrow.

In addition to inhibiting the access of ATGL to CGI-58, it is now believed that FSP27 affects ATGL-mediated lipolysis in adipocytes by: a) regulating hydrolase activity of ATGL, b) regulating expression and distribution of G0S2, and/or c) that FSP27 depletion causes fragmentation of lipid droplets which increases the surface area of lipid droplets, thus increasing the access of lipases.

The effect of FSP27 on TG hydrolase activity of ATGL is determined by the following protocol: HeLa cells stably expressing or non-expressing FSP27 are transfected with ATGL and CGI-58. The cell homogenates are incubated with ³H-labeled triolein as substrate and its hydrolysis is measured. As described in FIGS. 1A-1D, G0/S1 switch gene, G0S2, regulates ATGL-mediated lipolysis via inhibiting its TG hydrolase activity. The distribution of G0S2 is mostly cytosolic but under stimulated conditions a small percentage of it distributes to LDs. Therefore, the effect of expression of FSP27 or its functional domain(s) on the expression and distribution of G0S2 under both basal and/or stimulated conditions in human adipocytes is examined.

### FSP27 Depletion Increased Both Basal and Stimulated Lipolysis In Human Adipocytes.

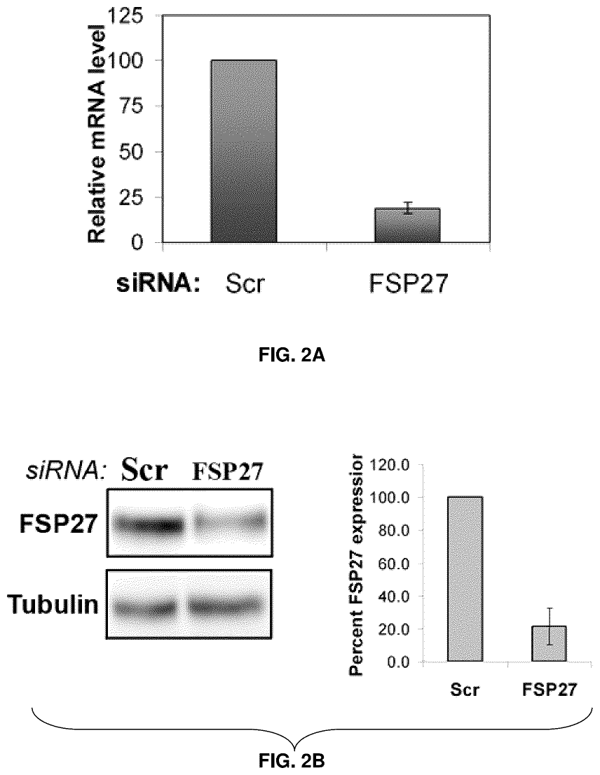

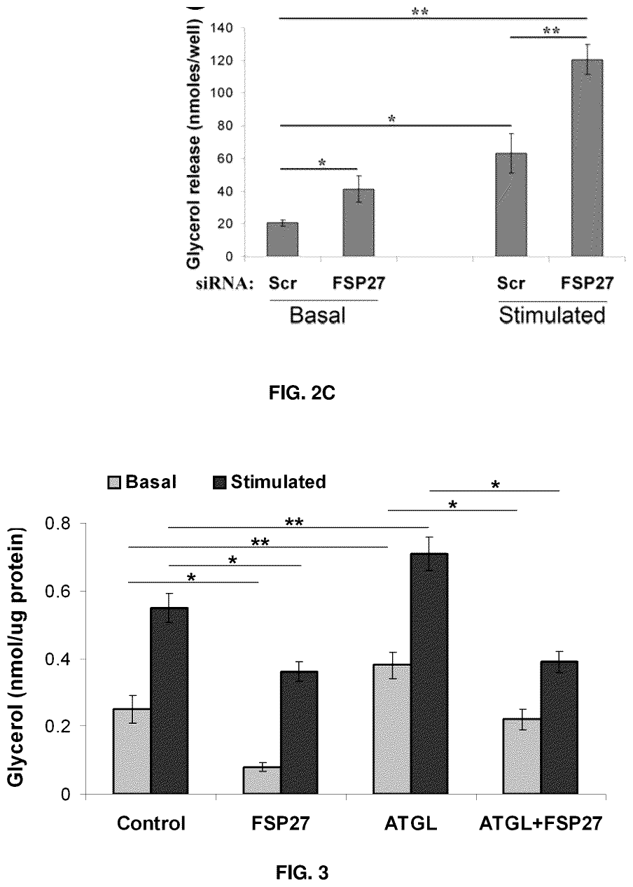

Non-specific scrambled (Scr) siRNA was used as a control in all experiments. FIG. 2A shows the relative mRNA levels in siRNA-transfected human adipocytes. FIG. 2B shows the immunoblot and quantification of protein expression levels of FSP27 and β-tubulin (loading control) of siRNA-transfected human adipocytes. FIG. 2C shows the biochemical quantification of basal and stimulated lipolysis based on measurement of glycerol release after 2 hours. Values are means ±standard error; *p<0.0 and **p<0.001, n=3 (unpaired t-test).

### FSP27 expression decreased ATGL-mediated lipolysis.

FIG. 3 shows glycerol released in cell culture media from human adipocytes expressing EGFP, FSP27-HA and/or ATGL. Control cells were infected with EGFP-containing empty virus. Glycerol released in 2.5 h was measured and normalized to total protein. Values are means ±standard error; *p<0.001 and **p<0.05, n=3 (unpaired t-test).

### FSP27 negatively regulates ATGL expression and lipolysis in human adipocytes.

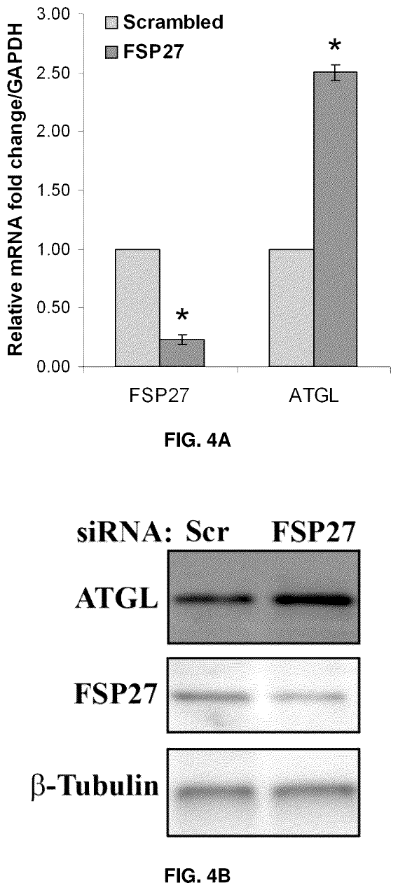

Human adipocytes were cultured and differentiated. FIG. 4A shows where RNA was extracted from control and siRNA-treated adipocytes, and mRNA levels were measured by quantiative PCR and normalized by GAPDH mRNA. The data show an average of three independent experiments. FIG. 4B shows protein lysates from control and siRNA-treated adipocytes were loaded at 15 μg/lane and probed with antibodies against FSP27, ATGL or β-tubulin. Image is representative of at least three independent experiments.

### FSP27 inhibits ATGL gene transcription (promoter/enhancer) activity via Egr1.

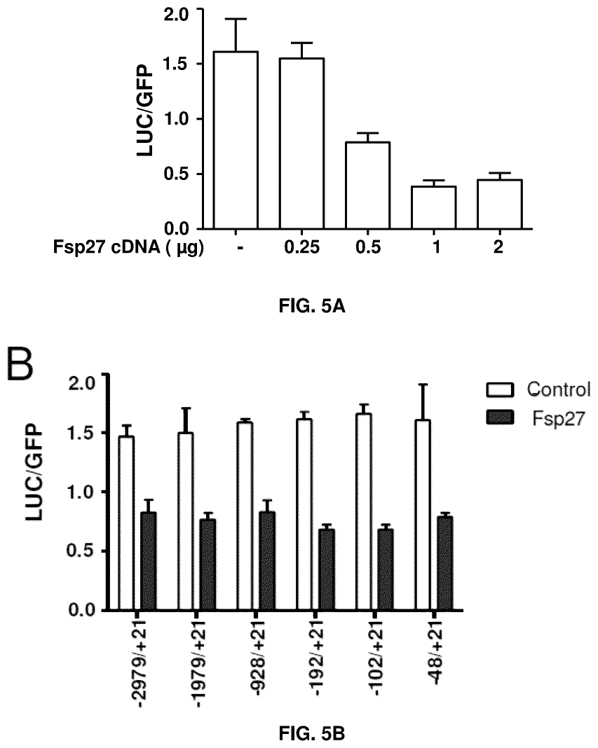

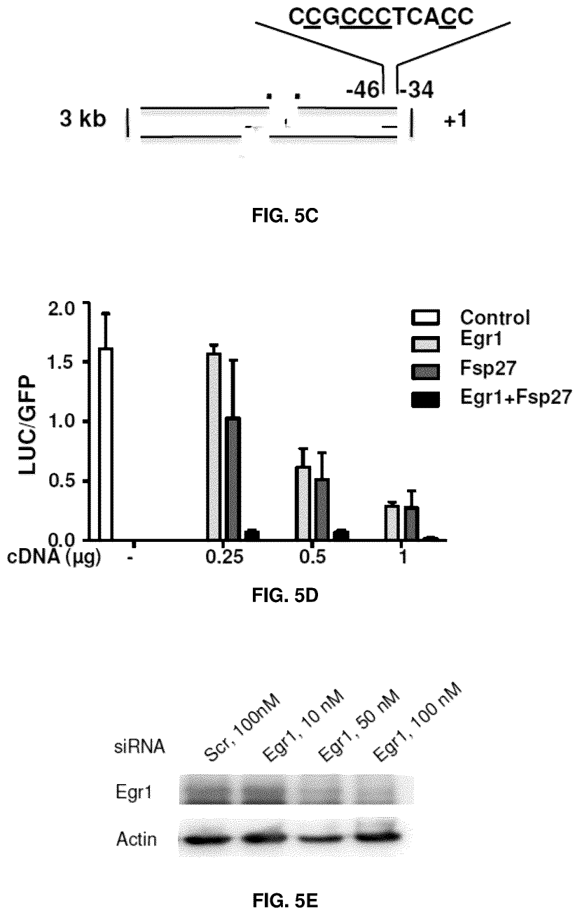

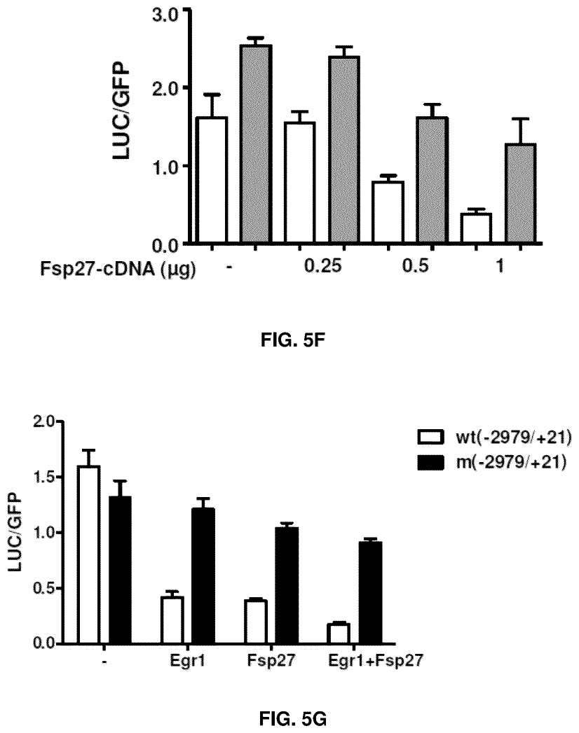

FIGS. 5A, 5B, 5D, 5F, and 5G show where HEK293T cells cultured in 12-well dishes were transfected with the full length (−2979/+21), C→T mutated, or truncated ATGL luciferase promoter constructs, cDNA for eGFP; cDNAs for FSP27 and Egr1 as well as scrambled siRNA and Egr1 siRNA as indicated. After 48 h, cells were washed three times in cold PBS and harvested in the reporter lysis buffer. Luciferase activity in cell lysates was assayed and normalized by eGFP fluorescence. Data are presented for triplicate samples as mean+SD; * p<0.05 as estimated by unpaired t-test.

FIG. 5D shows the synergistic effect between Egr1 and FSP27 with p<0.05. Experiments were repeated at least 3 times (FIGS. 5A, 5B, 5D, 5G) and 2 times (FIG. 5 F).

FIG. 5C is a schematic representation of the proximal region of ATGL promoter with the consensus Egr1 binding site. Nucleotides that have been chosen for the site-directed mutagenesis are underlined.

FIG. 5E shows HEK293T cells growing in 35 mm dishes were transfected with scrambled or Egr1 siRNA. Cell lysates were collected 48 h post-transfection, separated by 12.5% PAGE and immunoblotted with Egr1 and actin antibodies. The experiment was repeated at least 2 times.

### FSP27 Protected Human Adipocytes Against FFA-Induced Insulin Resistance.

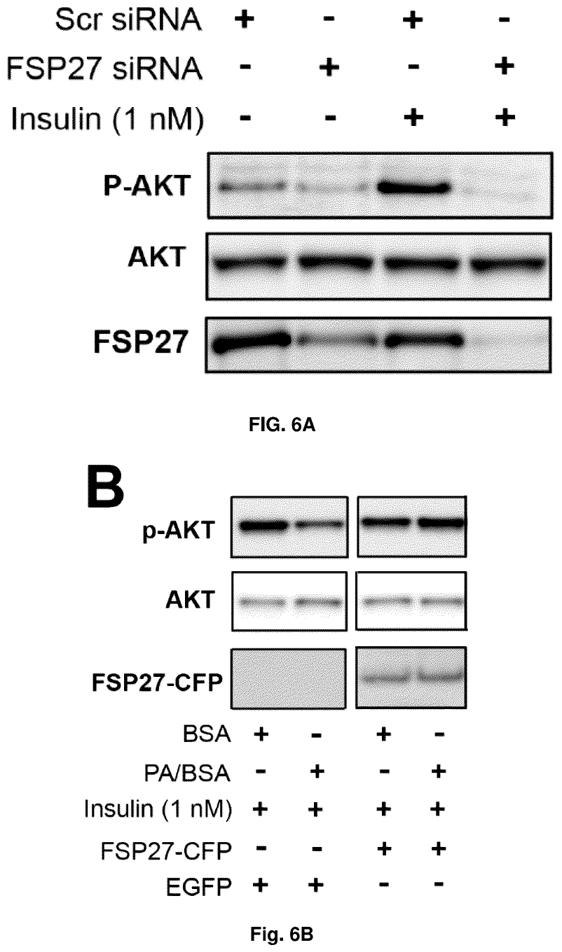

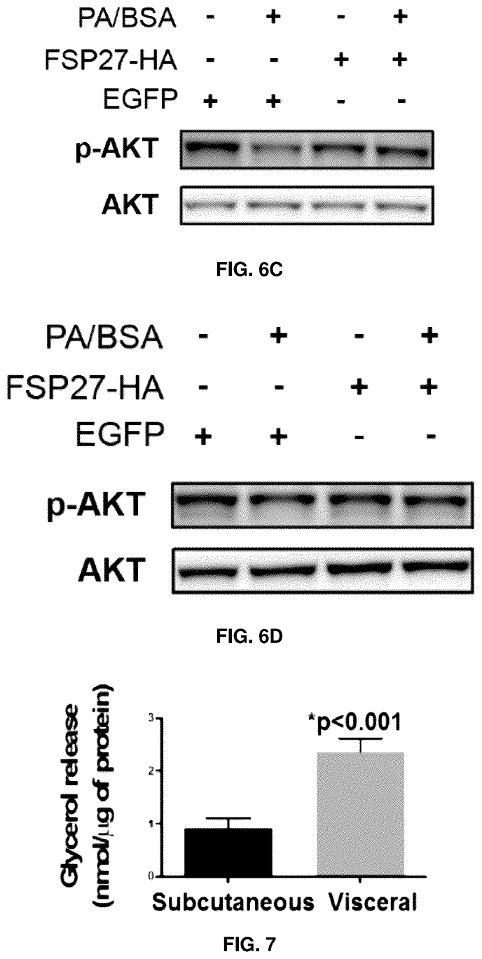

FIG. 6A shows where insulin stimulated AKT phosphorylation in human adipocytes after siRNA-mediated FSP27 knockdown. FIG. 6B shows insulin stimulated AKT phosphorylation in human adipocytes after overnight treatment with 100 μM PA/BSA in the presence or absence of FSP27-CFP or EGFP (Control). FIG. 6C shows FSP27-HA expression protects adipocytes differentiated from WT mouse embryonic fibroblasts (MEFs) against 100 μM PA/BSA-mediated inhibition of insulin stimulated AKT phosphorylation. FIG. 6D shows 100 μM PA/BSA or FSP27-HA expression had no effect on insulin stimulated AKT activation in adipocytes differentiated from ATGL-KO MEFs.

### FFAs Impairs Insulin Signaling and Promotes Insulin Resistance in Human Primary Adipocytes.

FSP27 depletion in human adipocytes increased ATGL expression and lipolysis and hence increased FFA levels, and it was determined whether FSP27 depletion affects insulin induced signaling. Indeed, FSP27 knockdown decreased insulin-mediated stimulation of AKT phosphorylation (FIG. 6A). Also, FSP27 overexpression protected human adipocytes against FFA-induced insulin resistance (FIG. 6B).

Furthermore, adipocytes derived from ATGL-KO MEFs but not WT were resistant to FFA-induced insulin resistance (FIG. 6C and FIG. 6D).

FIG. 7 shows that basal lipolysis was significantly higher in visceral depots compared to subcutaneous. Glycerol release was measured in 12 subcutaneous, 15 omentum adipose depots and normalized to total μg of protein. Data are presented as ±SEM.

### Increased Lipolysis in Visceral Adipose Negatively Correlates with FSP27 Expression.

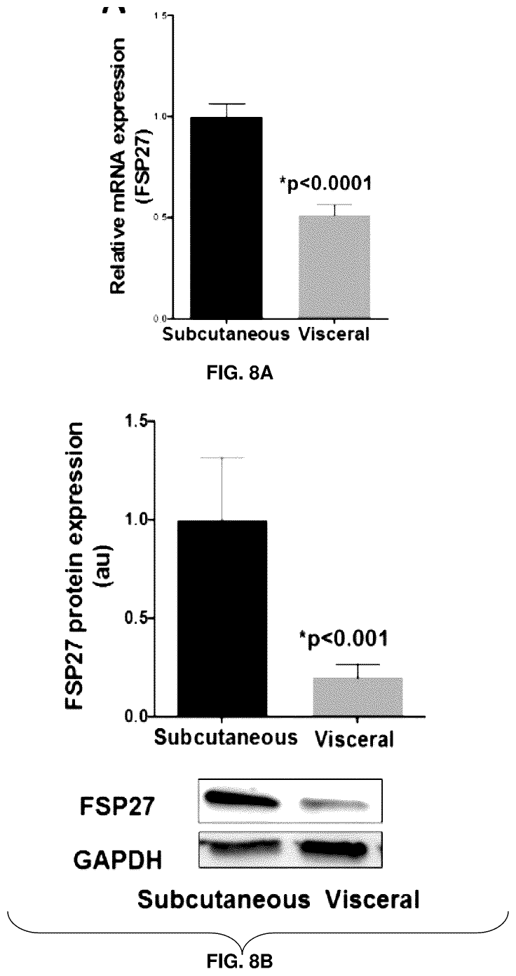

FIG. 8A shows that basal FSP27 was significantly higher in subcutaneous depot. FSP27 mRNA was measured in 27 paired samples from subcutaneous and omentum depots. Data are presented as ±SEM. FIG. 8B shows basal FSP27 protein was measured in 13 paired subcutaneous, omentum depots. Data are presented as ±SEM.

### Effect of FSP27 Depletion on Insulin Signaling in Adipocytes.

Akt activation is regulated by PIP3, and PIP3 levels are tightly regulated by phosphatidylinositol (PI)-3K and phosphatases, such as PTEN, which antagonizes PI3K/Akt signaling by dephosphorylating PIP3. The phosphorylation of PTEN is measured at Ser³⁸⁰/Thr³⁸²/Thr³⁸³. Also, the phosphorylation of IRS-1 and AS160 is measured and compared with the expression of FSP27.

FSP27 expression was lower in visceral adipose depot of a cohort of obese human subjects, as shown in FIGS. 8A-8B. The phosphorylation of IRS-1 and AS160 is measured in both visceral and subcutaneous adipose tissue of obese human subjects and compared with FSP27 expression

FIGS. 9A-9B show that siRNA-mediated FSP27 knockdown increases lipolysis and impairs insulin signaling. FIG. 9A shows knockdown of FSP27 in subcutaneous adipose tissue increased rate of glycerol release in the media. Data are presented as ±SEM (n=7). FIG. 9B shows siRNA-mediated FSP27 depletion decreased Akt phosphorylation.

The involvement of activated JNK and p38 stress pathways in FSP27-depleted inhibition of Akt in adipocytes and muscle and liver cells. FSP27 depletion increases lipolysis and FFA release in human adipocytes. Since FFAs increase ceramide content, which has been shown to activate MLK3, the upstream kinase of JNK and p38 pathways, the activation of these pathways. JNK and p38 are activated by phosphorylation at Thr¹⁸³/Tyr¹⁸⁵ and Thr¹⁸⁰/Tyr¹⁸², respectively, FSP27 depletion increases lipolysis and FFA release in human adipocytes. Since FFAs increase ceramide which activates MLK3, an upstream kinase for JNK and p38. Phosphorylation of JNK and p38 by MLK3 at Thr¹⁸³/Tyr¹⁸⁵ and Thr¹⁸⁰/Tyr¹⁸², respectively, causes downregulation of insulin receptor signaling in adipose, muscle and liver cells to promote insulin resistance.)

### Recombinant FSP27 Improves Insulin Signaling in Visceral Adipose.

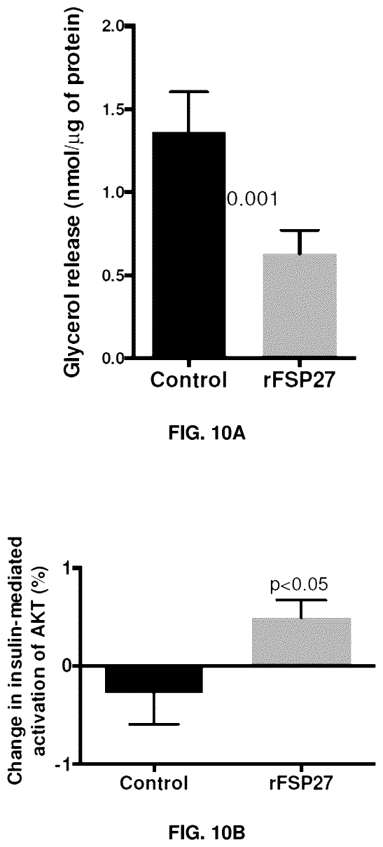

FIG. 10A shows the treatment of visceral depot with recombinant FSP27 decreased basal lipolysis. FIG. 10B shows the quantification of insulin-stimulated AKT phosphorylation. Data are presented as ±SEM. *p<0.05.

### FSP27 (120-220) Protects Against FFA-Induced Insulin Resistance in Human Adipocytes.

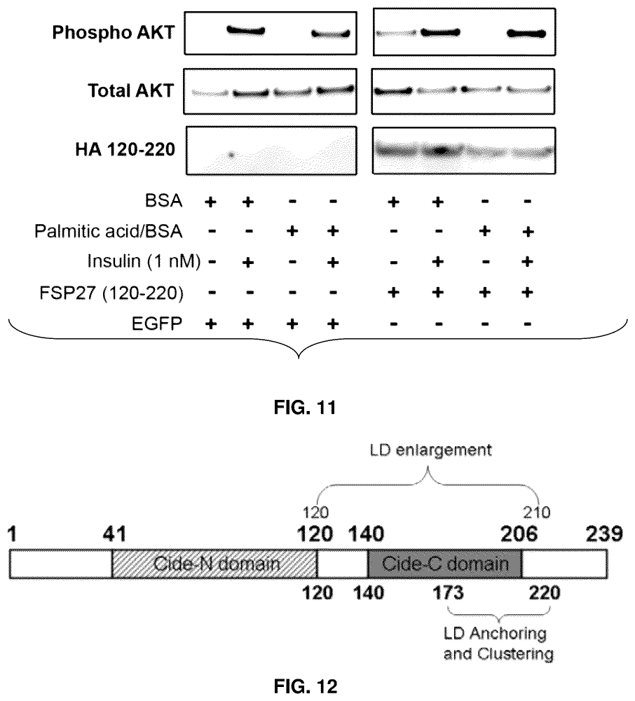

Insulin stimulated AKT phosphorylation in human adipocytes. The core FSP27 domain that is associated with TG accumulation, aa 120-220, was expressed using lentivirus, with EGFP as a control. The cells were treated overnight with 100 μM PA/BSA. The blots in FIG. 11 show AKT phosphorylation in basal and insulin stimulated conditions. FSP27 (120-220) protected human adipocytes from inhibition of AKT phosphorylation by exogenous PA.

FIG. 12 shows the schematic representation of FSP27 and its functional domains: CIDE-N terminal domain and CIDE-C terminal domain. The N-terminus of FSP27 is from amino acids 1-120 and the C-terminus is from amino acids 121-239. FSP27-mediated enlargement of lipid droplets (LDs) consists of two independent steps, clustering followed by fusion of LDs. Amino acids 172-210 are necessary and sufficient for FSP27-mediated clustering of LDs. The clustering of LDs has no effect on their size and cellular TG levels. The LD clustering is followed by their enlargement. Amino acids 120-210 are sufficient for clustering and enlargement of LDs.

FIG. 13 shows that the FSP27 sequence is conserved in vertebrates; for example, >90% conserved sequence in FSP27 in humans, mouse, monkey, dog, cow and frog.

### Generation of Adipose Specific Human FSP27-Overexpressing Transgenic Mice.

FSP27-mediated suppression of lipolysis in adipose tissue can protect from insulin resistance and Type 2 diabetes.

FSP27 was cloned in ROSA26-CMV-loxSTOPlox vector and mice generated conditionally

over-express FSP27. These mice are crossed with Adipoq-cre mice to specifically over-express FSP27 in adipose tissue (AT), (AT-FSP27tg). Based upon the data shown in FIGS. 6A-6D, a 2-3 fold increase in FSP27 expression in WAT it is now believed to be sufficient to provide a protective effect of FSP27 against high Fat Diet (HFD)-induced insulin resistance.

In order to determine whether overexpressing FSP27 also alters other proteins associated with lipid droplet protection and lipolysis, PLIN1, ATGL and HSL protein and/or phosphorylation are analyzed under basal and stimulated states; then followed with identifying any alterations in adipocyte lipolysis. Fat deposition and lipid metabolites in muscle and liver are measured in the experimental animals. To estimate intracellular pools of FSP27, lipid droplet pools of FSP27 are isolated and quantified. The amount of FSP27 is expressed relative to the number and/or surface area of adipocytes.

### Glucose Homeostasis and Metabolic Phenotyping:

The littermates are subjected to physiological characterization at 6, 12 and 26 weeks. Body weights and body composition are measured by Magnetic Resonance Imaging. Under both fed and fasted conditions, level of circulating, glucose, insulin, FFA, glycerol, GH, IGF-1 and adipokines (leptin, reisistin, and adiponectin) are determined with commercially available ELISA or enzymatic kits. Intraperitoneal glucose tolerance testing (GTT) (2 g/kg body weight), and intraperitoneal insulin tolerance test (ITT) (0.75-1.25 U/kg body weight) are assessed. Separate cohorts of mice are placed on either a low (10%) or high (60%) fat diet (HFD) (D12450B and D12492, Research Diets) for 12 wks and studied in a similar manner. Changes in metabolic rate and energy expenditure are measured by the Comprehensive Lab Animal Monitoring System (CLAMS, Columbus Instrument). Mice are acclimated for 24 h then monitored for a 48 h fed period followed by a 24 h fasted period. Activity (determined by infrared beam breaks), food intake, energy expenditure (normalized to lean body mass) and heat production are also measured. Respiratory exchange ratio (RER) (VCO2/VO2) is calculated from the gas exchange data for light and dark phases. After completion of the physiological assessment, mice are sacrificed. Blood, skeletal muscles (quadriceps, EDL, and tibialis anterior), liver, heart, kidney, subcutaneous (SC), perigonaldal (PG), and brown fat depots, brain, and pancreas and/or isolated islets are collected and weighed. Tissues are prepared for histology and mRNA and protein is extracted for further analysis.

### Insulin Signaling, Glucose Uptake, and Lipolysis:

Results from the hyperinsulinemic-euglycemic clamp and ITT are confirmed by assessing insulin signaling in insulin sensitive tissues, including the adipose tissue (AT), muscle, and liver. Fifteen minutes after intravenous injection of insulin, tissues are isolated and insulin signaling intermediates (e.g. IR, IRS1, Akt, PI3K, and mTORC1 and, where relevant, their phosphorylated counterparts) are assayed by western blot analysis. For measurements of glucose uptake and lipolysis from primary adipocytes, fat pads are enzymatically digested. Lipolytic rate is quantified by glycerol and FA release, and glucose uptake is determined by uptake of [³H] 2-Deoxy-D-glucose. Since adipocyte size is believed to vary in these mouse models, we will quantify lipolysis “per adipocyte” and “per unit adipocyte surface area” are quantified.

### Histology of Adipose Tissue, Ectopic Lipid and Cytokine Quantitation:

AT histology is conducted to examine the adipocyte morphology and macrophage infiltration. Adipocyte size and macrophage infiltration are quantified. Ectopic fat deposition in muscle and liver is visualized by histology and triacylglycerol, diacylglycerol, and ceramides are quantified. The circulating levels and mRNA expression of pro-inflammatory cytokines (IL1β, TNFα, IL6) are determined, as FFAs have been shown to modulate macrophage activation and the expression of pro-inflammatory cytokines. Furthermore, TNFa has been shown to increase lipolysis in a FSP27 dependent manner.

### Protected Insulin and Glucose Response in High-Fat Fed Adipose Tissue-Specific Human-FSP27 Transgenic Mice (AT-hFSP27tg).

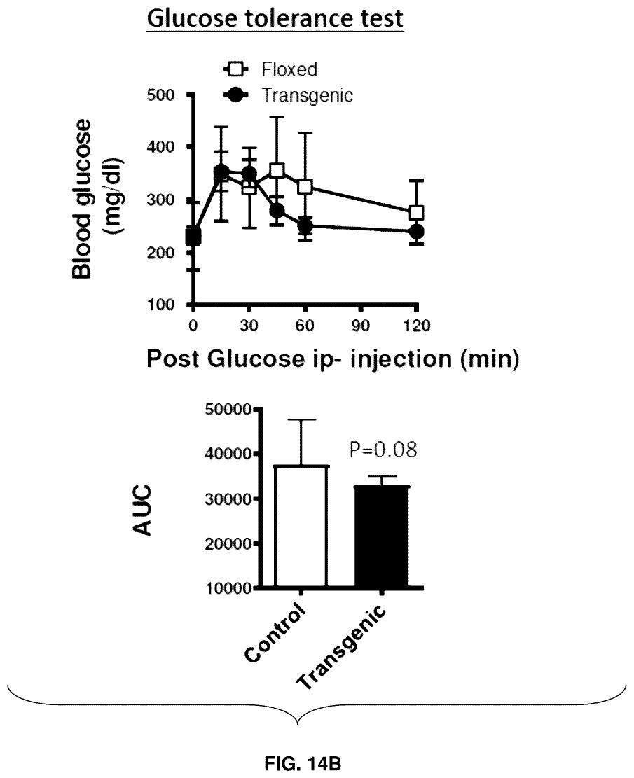

FIG. 14A shows insulin tolerance test (ITT), while FIG. 14B shows glucose tolerance test (GTT) in AT-hFSP27tg mice. Males (5-month-old, n=3/group) were fed a 60% HF for 3 months. Data: mean ±SEM. Plots at the bottom of the curves show area under the curves. These data show that overexpressing FSP27 in adipocytes prevents diet-induced insulin resistance.

### FSP27 knockout (FSP27^{-/-}) are Glucose and Insulin Intolerant.

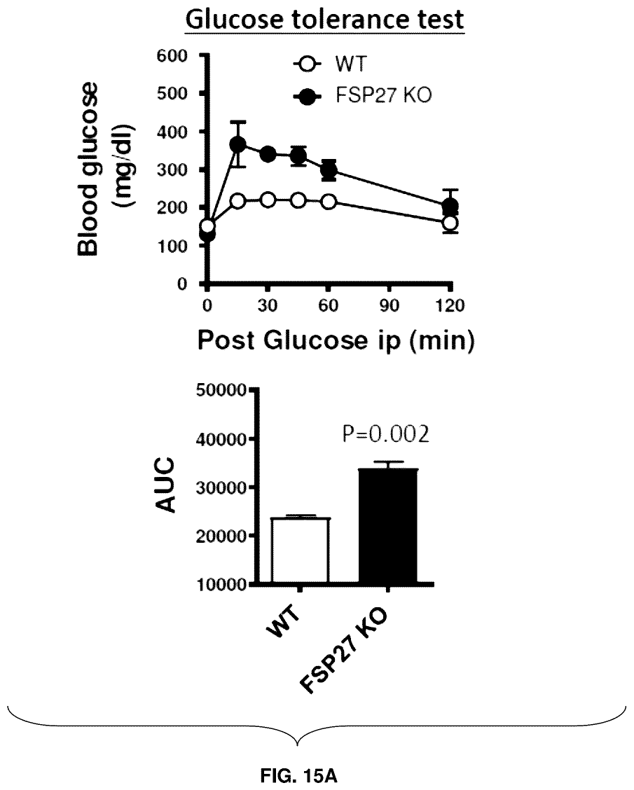

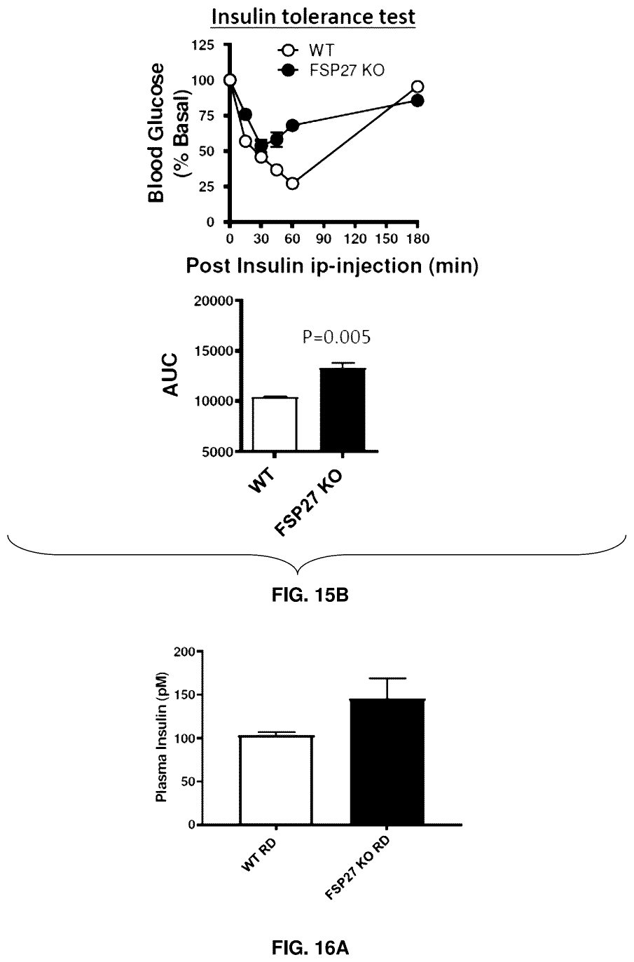

FIG. 15A shows glucose tolerance test (GTT), while FIG. 15B shows insulin tolerance test (ITT) in FSP27^{-/-} mice. Males (4 month-old; n=3/group) Data: mean+SEM; p<0.05 in Fsp27^{-/-} vs Wild type (WT) mice. Plots at the bottom of the curves show area under the curves.

### Plasma insulin and NEFA in FSP27^{-/-} Mice on a Regular Diet (RD)

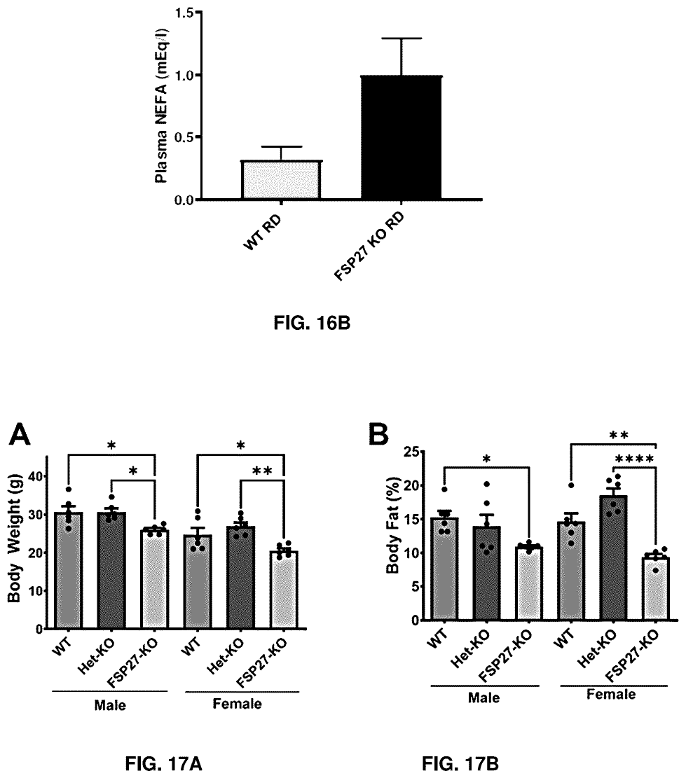

Fasting blood insulin and non-esterified fatty acid (Free fatty acids) levels were higher in FSP27 knockout mice, as shown in FIG. 16A and FIG. 16B.

### EXAMPLE 2:

Example 2 shows a novel role of Fsp27 in muscle performance. Fsp27^{-/-} and Fsp27^{-/-} mice, both males and females, had severely impaired muscle endurance and exercise capacity compared to wild-type controls. Liver and muscle glycogen stores were similar amongst all groups fed or fasted, and before or after exercise. Reduced muscle performance in Fsp27^{-/-} and Fsp27^{-/-} mice was associated with severely decreased fat content in the muscle. Furthermore, results in heterozygous Fsp27^{-/-} mice indicate that Fsp27 haploinsufficiency undermines muscle performance in both males and females. In sum, these physiological findings reveal that Fsp27 plays a critical role in muscular fat storage, muscle endurance, and muscle strength.

### MATERIALS AND METHODS

### Mouse Maintenance, Generation and Dissection

Fsp27^{-/-} mice were procured from Dr. Yoshikazu Tamori, Kobe University, Japan. These mice were in C57BL/6N background. FSP27-KO mice were obtained by crossing of heterozygotes. The details of genotyping the heterozygous and homozygous mice were published previously. Crossing of Fsp27^{-/-} mice provided littermate control, Fsp27^{-/-} and Fsp27^{-/-} mice. Both male and female mice were fed regular chow diet. All mice were 8-10 months of age at the time of experimentation. Animals were housed at the Ohio University Animal Facility (Athens, OH) with access to mouse chow and water ad libitum and an automatic 12 hr day/night cycle. Experimental protocols with mice were performed in accordance with the IACUC guidelines. Ohio University is approved by the NIH-Office of Laboratory Animal Welfare (Animal Welfare Assurance number A3610-01). It also has a USDA license 31-R-0082, compliant with the USDA Animal Welfare Act Regulations and the Public Health Service Policy on Humane Care and Use of Laboratory Animals in accordance with the Guide for the Care and Use of Animals. Generation of the Fsp27^{-/-} mice was previously described. All animals were sacrificed by exposure to CO₂ followed by cervical dislocation. Collected tissues were snap-frozen in isopentane and stored at −80° C. Sera were stored at −20° C.

### Metabolic Profiling

Mice were housed individually at 22° to 23° C. in transparent plastic cages (31×20×13 cm) placed in a light-controlled (12-hour light/12-hour dark cycle). Mice were housed individually in metabolic chambers and acclimatized for 48 h. Over the subsequent 48 h, the metabolic parameters were measured every 15 min by a Comprehensive Lab Animal Monitoring System (CLAMS, Columbus Instruments). Oxygen consumption and carbon dioxide production of mice in the fed condition was measured with an indirect calorimetric system every 15 minutes for 24 hours at 28° C. Every chamber has a dedicated electrochemical oxygen sensor and NDIR carbon dioxide sensor for simultaneous measurements. Energy expenditure was calculated as the product of the calorific value of oxygen (3.815+[1.232×respiratory quotient]) and the volume of O2 consumed. Locomotor activity was recorded with an array of infrared photo beams that surround the cage. XY position of the animal was continuously recorded by the beams arranged in a grid pattern. Z-axis sensors were used to measure rearing events. Food intake was by indirect calorimetry. For food consumption center feeder containing powdered chow was used. Water consumption was monitored by Volumetric Drinking Monitor system. Body composition (fat mass) was determined by NMR (LF50 BCA-Analyzer, Bruker) in live mice.

### Lipid Analysis and Glycogen Determination

Serum from WT and Fsp27^{-/-} mice (n=5) was analyzed by the Lipid Core at Vanderbilt University Medical Center for total FFA and TG as well as the lipid species composition. Liver and muscle glycogen content in fasted and unfasted WT and Fsp27^{-/-} mice (n=5), before and after exercise (n=5, male) was determined with the Glycogen Assay Kit (Sigma-Aldrich) per the manufacturer's instructions.

### Glucose Tolerance Test and Insulin Tolerance Test

Glucose tolerance test (GTT) was performed in 8 hrs fasted mice. 1.5 g/kg of glucose was injected intraperitoneally, and blood glucose was monitored at the indicated time points using a GE100 blood glucose monitoring system glucometer (GE Healthcare). Insulin tolerance test was performed in 6 hrs fasted mice. 0.75 IU/kg insulin (Humulin-N, Eli Lilly) was injected intraperitoneally and blood glucose was monitored, as above.

### Treadmill Running Endurance

Muscle endurance was determined by a muscle fatigue-test on the Exer 3/6 Metabolic Treadmill (Columbus Instruments). A fatigue zone was determined at the back end of the treadmill, which included the shock grid region and approximately one mouse body length of running track. Mice were trained and acclimated to use the treadmill at low speed (10-12 m/min) over a course of 3 days, with the speed increasing by 1m/min each day. Mice which received an abnormal number of shocks were removed from training and were not utilized for the test. Treadmill fatigue-testing was performed by placing the mice on the stationary treadmill and slowly increasing the speed to 12 m/min. The speed was then slowly increased to a maximum speed of 26 m/min. A total of 6 mice were tested simultaneously and allowed to run freely until spending 5 consecutive seconds in the fatigue zone (defined above) after which point the shock grid was disabled and the total distance run (km) by each mouse was determined and normalized to body weight (g).

Four-Limb Hanging Grid Test

Muscle strength was examined by four-limb hanging grid test with a custom made 38 cm by 30 cm grid with a 1 cm metal mesh. Mice were placed on the grid and allowed to explore the surface for 2 minutes before testing. The hanging grid was slowly inverted and placed onto an apparatus which suspended the mice approximately 42 cm above a soft cushion. Mice were allowed to hang upside-down until their muscles could no longer support their body weight. The total time that each mouse spent inverted was recorded. Mice were tested a total of 5 tests. If a mouse showed signs of non-performance (such as continually looking downward and then releasing from the grid intentionally), that mouse was removed from the study. Fsp27^{-/-} mice were significantly lighter than WT and Fsp27^{+/-} mice, so we accounted for this difference by converting hanging time into holding impulse. Holding impulse was calculated by converting the weight of the mouse (g) to Newtons and multiplying it by the hanging time (sec).

### Muscle Fiber Typing and Microscopy

Muscles (gastrocnemius, TA, EDL) were cut on a cryostat (Leica CM 1950) into 10 um sections and mounted on Superfrost Plus microslides (VWR). Staining was performed as described by Bloemberg and Quadrilatero, with some modifications. Immediately after cutting, slides were air-dried for 10 minutes. Sections were blocked with 10% goat serum and Mouse-on-Mouse blocking reagent (1:25, Vector Laboratories) in PBS for 1 hour at room temperature. Primary antibodies targeting myosin heavy chains Type I, Type lla, and Type Ilb (BA-D5, SC-71,and BF-F3; Developmental Studies Hybridoma Bank, University of lowa) were combined at a concentration of 1:200 per antibody in blocking buffer and applied to sections overnight at 4° C. Slides were washed 3 times in PBS for 5 minutes. Secondary antibodies targeting each specific primary antibody (BA-D5, Alexa Fluor 350 lgG_{2b}; SC-71, Alexa Fluor 488 lgG₁; BF-F3, Alexa Fluor 555 lgM; ThermoFisher Scientific) were combined at a concentration of 1:500 per antibody in blocking buffer and applied to sections for 1 hour at room temperature in the dark. Slides were washed 3 times in PBS for 5 minutes and then mounted in Vectashield mounting medium (Vector Laboratories). Confocal microscopy was performed on Nikon A1R (Nikon, Japan) confocal microscope using 60× oil immersion objective. Images were processed using Nikon software. Fiber percentage for each type of fibers was calculated based upon total number of fibers in the field of each microscopic image.

### RNA isolation, Reverse Transcriptase-Polymerase Chain Reaction (RT-PCR), and Quantitative (q)PCR Analysis

Total RNA was isolated from muscle with TRIzol (Ambion, Life Technologies, Cat # 15596019). RNA (1 μg) was used to make cDNA using a RevertAidRT kit (Thermo Scientific, Cat # K1691). qPCR was performed with PowerUp SYBR Master Mix (Applied Biosystems, Cat # 100029284).

### Western Blot

Total proteins were isolated from muscle using mammalian cell lysis buffer (Cell Signaling Technology, Cat #786-180) in the presence of protease inhibitor (Roche, Cat #11836153001) and phosphatase inhibitor (Roche, Cat #04906837001). Samples were centrifuged twice to isolate the clear protein fraction. Protein was estimated and an equal amount of protein (pg) was loaded and resolved on 10% PAGE and subsequently transferred to the PVDF membrane. The membrane was then blocked and probed with primary antibody overnight followed by washing three times with TBST buffer and incubation with secondary antibodies (diluted in 4% BSA in TBST) for the next two hrs. Finally, the blots were washed three times with TBST and developed using HRP substrate (Immobilon Western Chemiluminescent HRP substrate-Millipore, Cat #WBKLS0500) and visualized under chemiluminescence. Images were captured using BioRad Imager and blots were quantified using ImageJ (NIH) software.

### Blood Lactate Measurement Blood lactate was measured before and after exercise by tail bleed using lactate meter (Nova Biomedical).

### RESULTS of Example 2

### Metabolic Characterization of Fsp27^{-/+} and Fsp27^{-/-} -mice

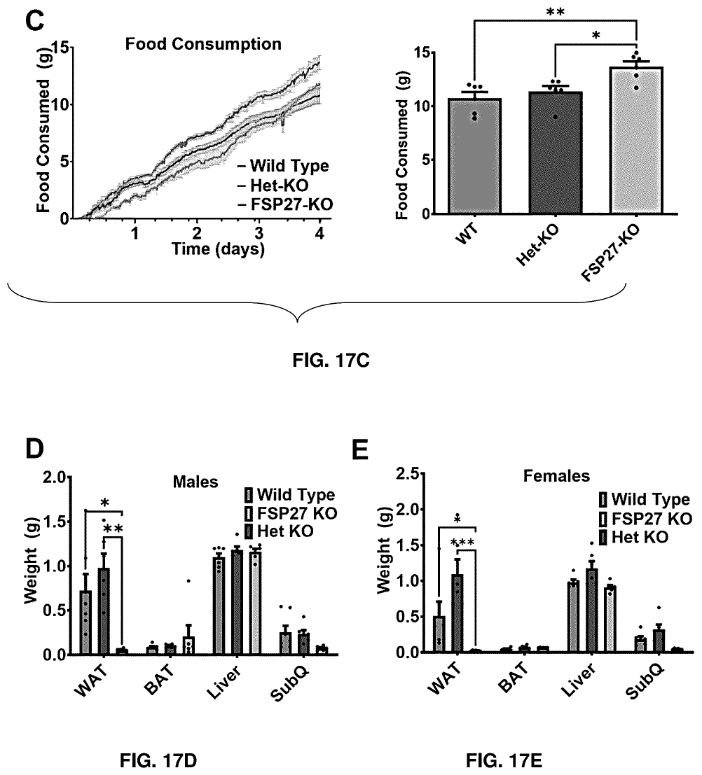

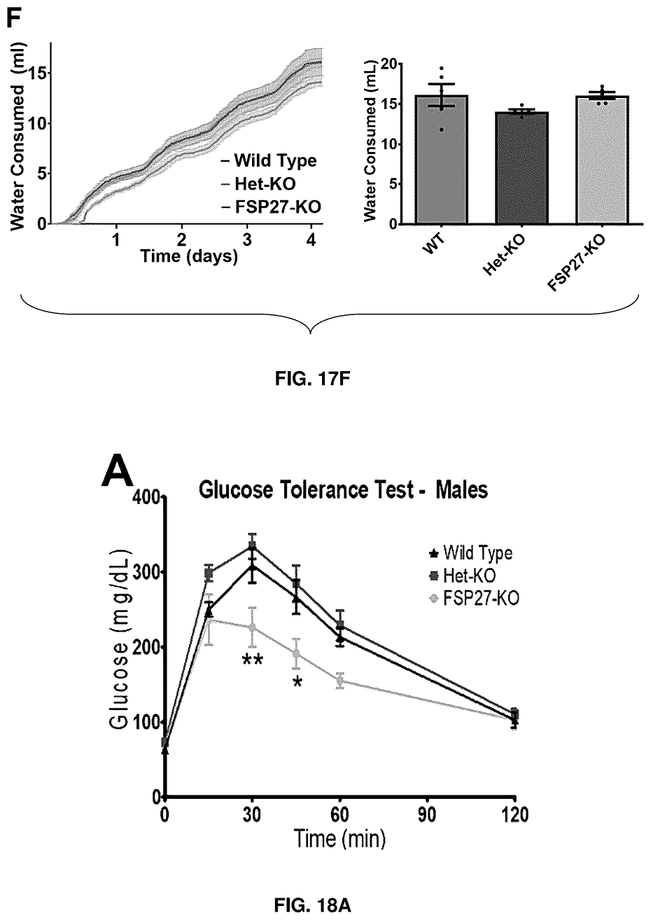

No significant difference in body weight or body fat were found between WT and Fsp27^{+/-} mice (FIGS. 17A and 17B). However, both male and female Fsp27^{-/-} mice showed significantly reduced body weight than WT or Fsp27^{-/-} mice (FIG. 17A).

Male Fsp27^{-/-} mice retained significantly less body fat than WT mice (FIG. 17B), while female Fsp27--mice retained less body fat than both WT and Fsp27^{+/-} mice (FIG. 17B).

Body weight and body fat of Fsp27^{+/-} mice did not differ from WT mice (FIGS. 17A, 17B). Fsp27^{-/-} mice showed increase food uptake (FIG. 17C).

Gonadal white adipose tissue (WAT) in both male and female Fsp27^{-/-} mice was significantly reduced compared to WT and Fsp27^{-/-} mice (FIGS. 17D and 17E). Although, subcutaneous (SubQ) fat was also diminished in both male and female Fsp27^{-/-}, it did not show significance, while brown adipose tissue (BAT) and liver mass remained unchanged (FIGS. 17D, 17E). Water consumption remained similar between WT, Fsp27^{+/-} and Fsp27^{-/-} mice (FIG. 17F). These data demonstrate that a single allele of Fsp27 is sufficient to maintain triacylglycerides (TAGs) levels in WAT and that complete loss of Fsp27 is necessary to reduce fat mass and increase food uptake significantly.

### Glucose and Insulin Tolerance were Improved in Fsp27^{-/-} -mice, but not in Fsp27^{+/-} mice

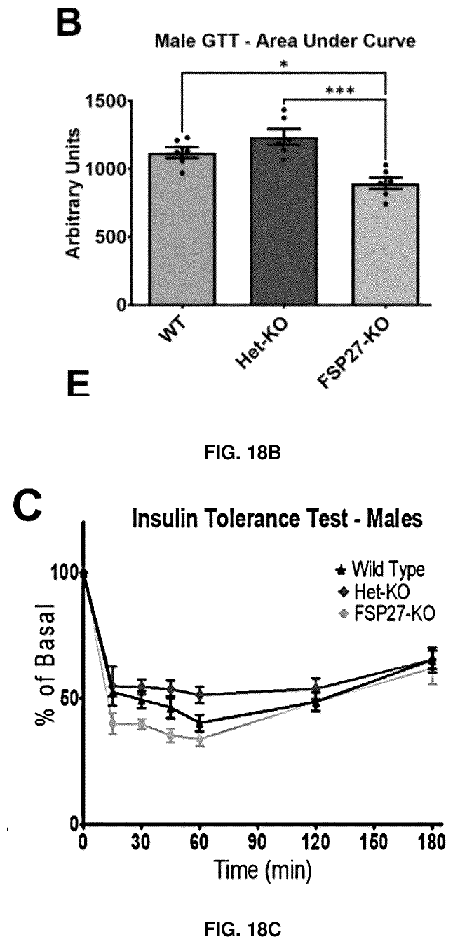

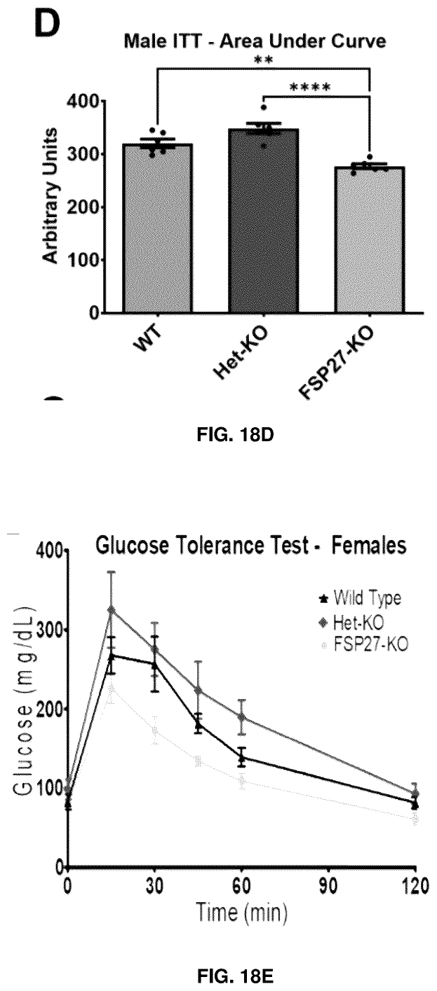

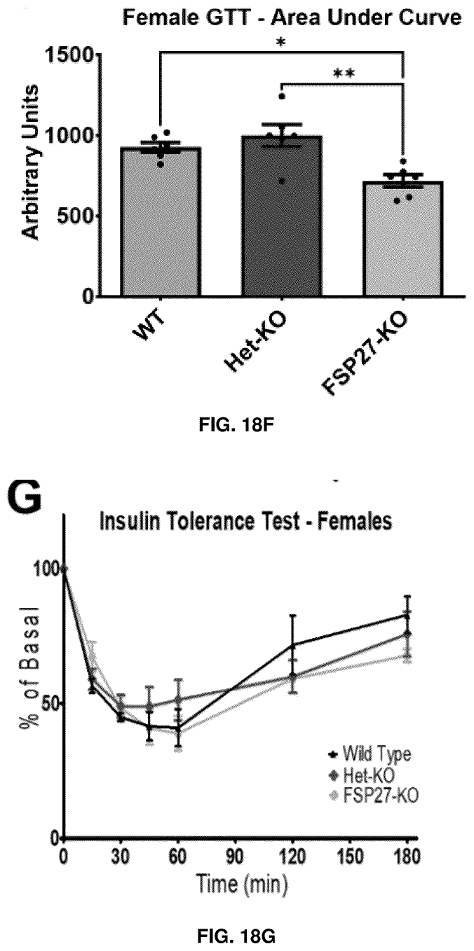

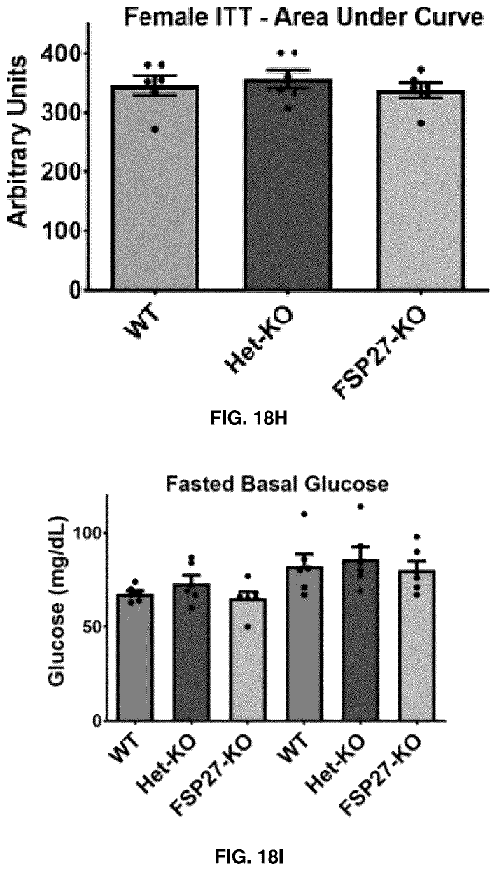

Glucose homeostasis was tested by intraperitoneal glucose tolerance test (GTT) and intraperitoneal insulin tolerance test (ITT) in 8-10-month-old mice. Male Fsp27^{-/-} mice were more glucose tolerant and insulin sensitive than WT and Fsp27^{+/-} mice (FIGS. 18A-18), while female Fsp27^{-/-} mice showed improvement in GTT, but not insulin sensitivity (FIGS. 18E-18H). No significant difference was observed in fasting blood glucose in male or female mice of either group (FIG. 181). Overall, the results identify that Fsp27^{-/-} mice are more glucose and insulin tolerant than WT or Fsp27^{+/-} mice.

### Fsp27′ Mice Have Higher Energy Expenditure, while Fsp27^{+/-} and Fsp27′ Mice Had Reduced Locomotor Activity at Night

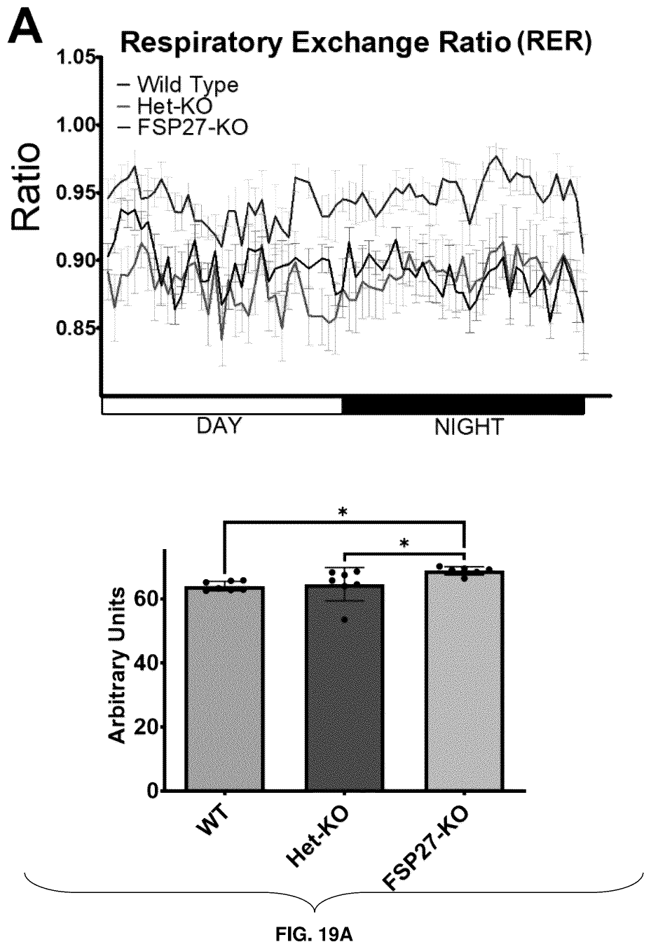

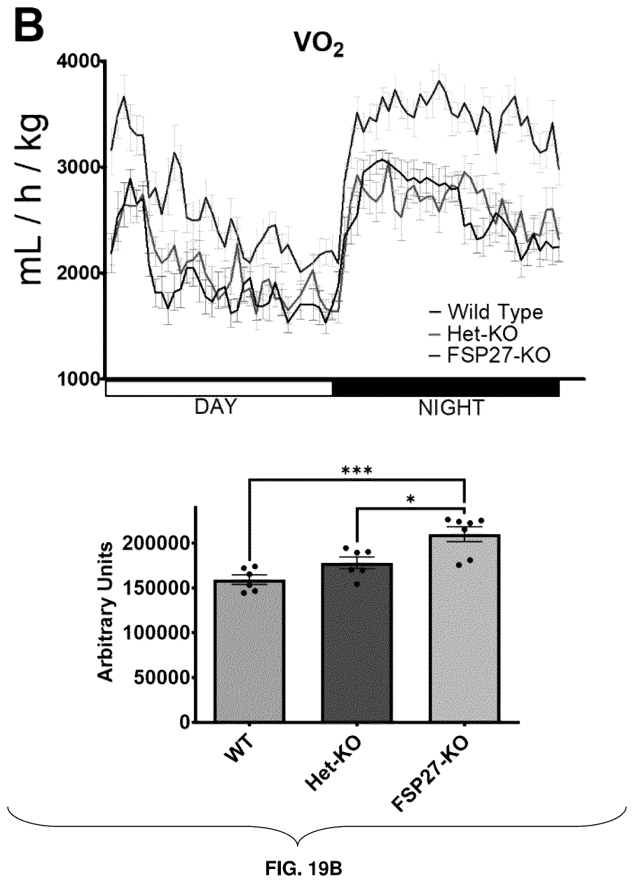

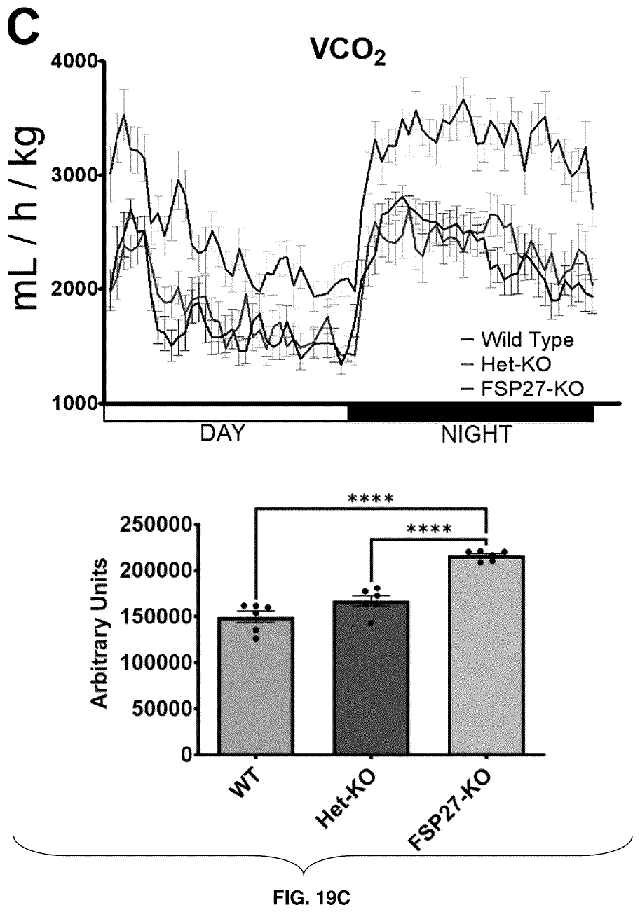

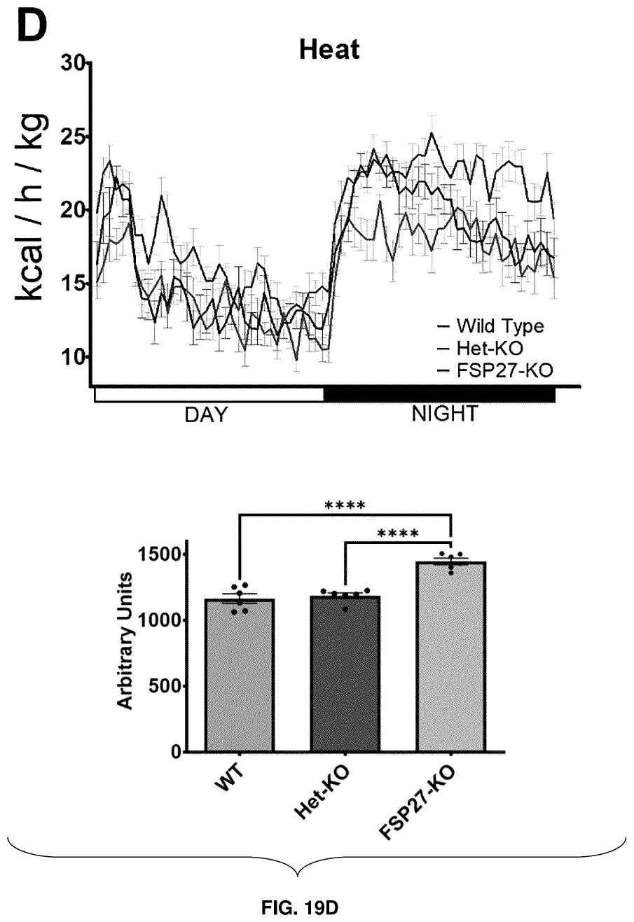

Energy expenditure and activity were assessed in metabolic cages over a 5-day period. There were no discernable differences in Respiratory Exchange Ratio (RER), Oxygen uptake (VO₂; maximum oxygen consumption), Carbon Dioxide Output (VCO₂; carbon dioxide production), and heat released between Fsp27^{+/-} and WT mice (FIGS. 19A-19D).

In contrast, we observed a significant increase in VO₂ in Fsp27^{-/-} mice, suggesting an increased metabolic rate (FIG. 19B). Similarly, VCO₂, respiratory exchange ratio, and heat released were also increased in Fsp27^{-/-} mice compared to WT (FIGS. 19A, 19C, 19D).

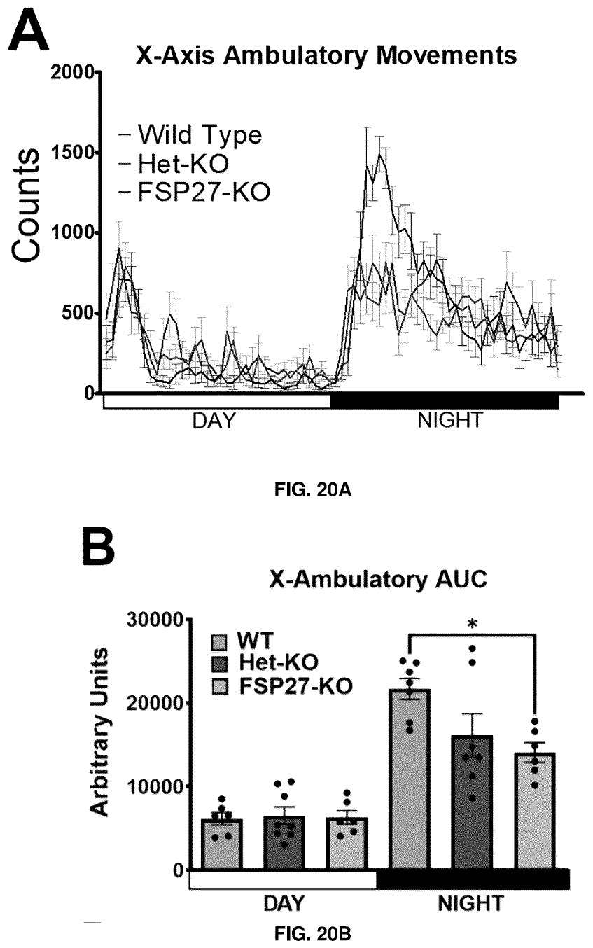

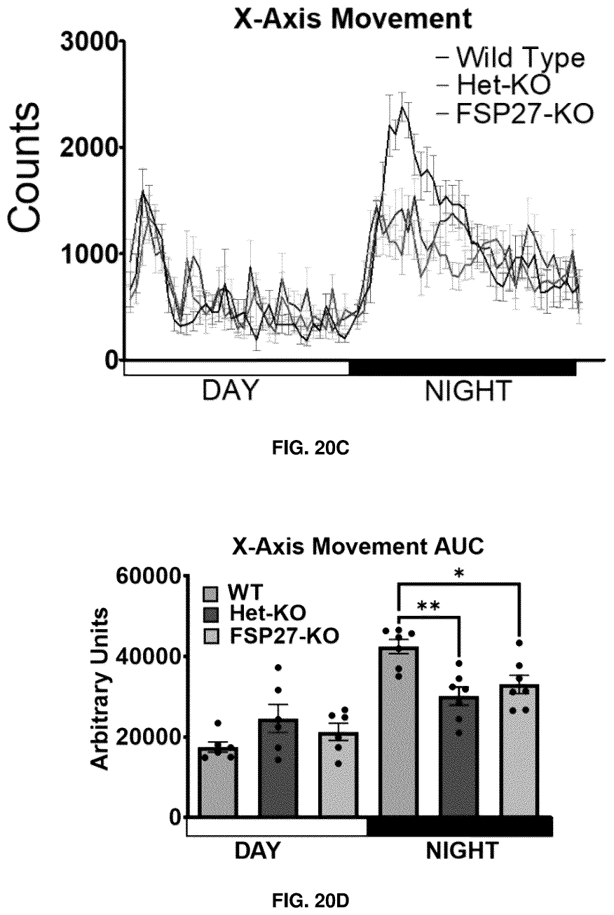

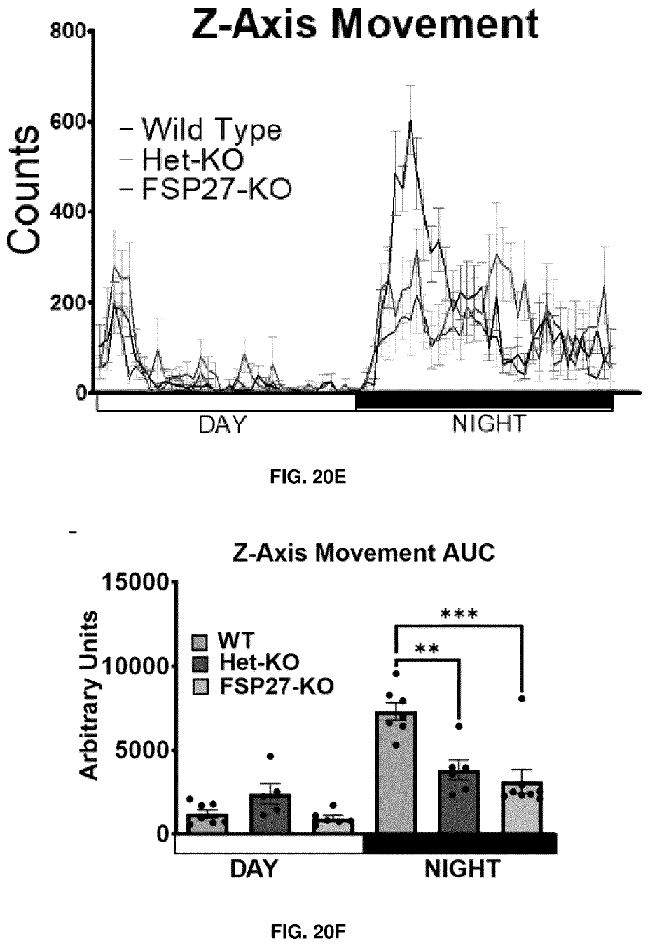

There were no observed differences in locomotor activity between the mice during the day cycle (FIGS. 20A-20F). However, during the night cycle, Fsp27^{+/-} mice showed reduced activity compared to WT mice in X-ambulatory movements (FIGS. 20A, 20B) as well as overall movement on the X-and Z-axis (FIGS. 204C-20F).

Likewise, Fsp27^{-/-} mice exhibited reduced activity compared to WT and to Fsp27^{+/-} mice on X-and Z-axis at night (FIGS. 20C-20F).

### Muscle Performance is Compromised in Fsp27^{+/-} and Fsp27^{-/-} Mice

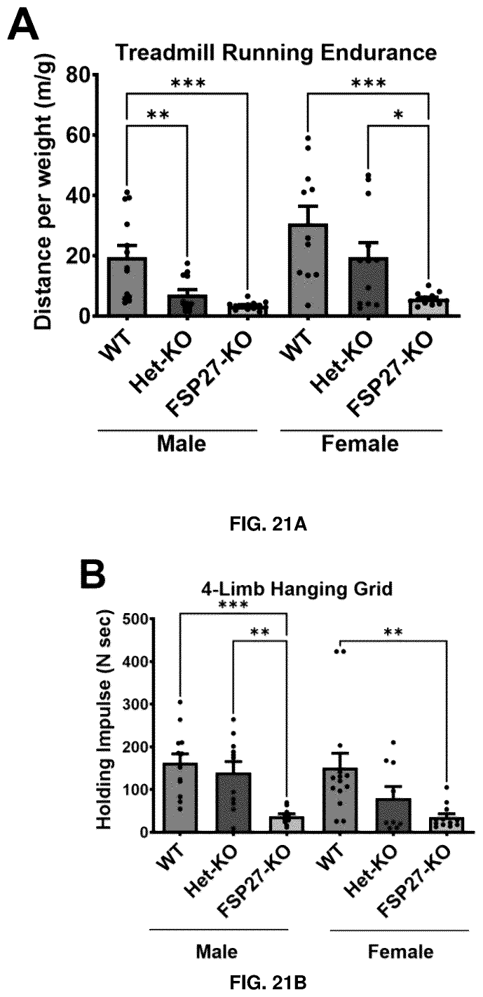

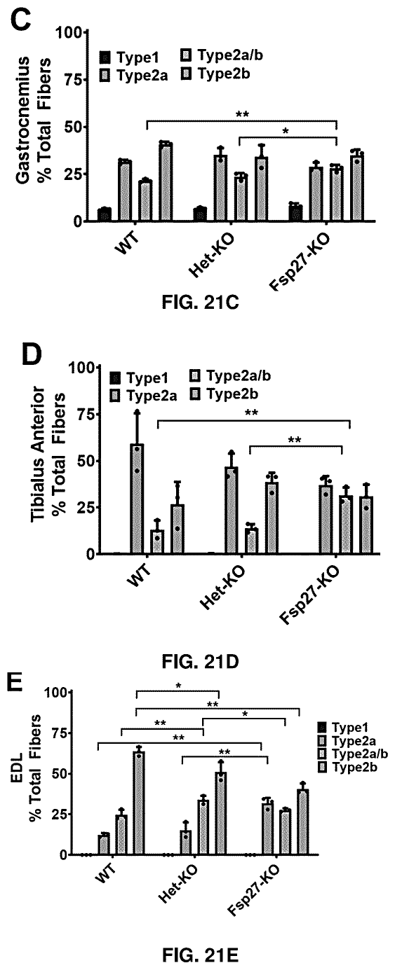

Treadmill running test and four-limb hanging grid test were used to measure exercise capacity and endurance. Both Fsp27^{+/-} and Fsp27^{-/-} male mice fatigued on the treadmill at a significantly shorter distance than WT mice (FIG. 21A). Fsp27^{-/-} males fatigued significantly earlier than Fsp27^{+/-} mice (FIG. 21A).

Similar results were obtained when data was calculated as distance per body weight (not shown). Both WT and Fsp27^{+/-} male mice outperformed Fsp27/mice on the four-limb hanging grid test (FIG. 21B). Female mice showed a similar trend, namely-WT and Fsp27^{+/-} mice outperformed Fsp27^{-/-} mice in endurance running (FIG. 21A), while only WT mice outperformed Fsp27^{-/-} mice on the strength test.

The fiber type of the gastrocnemius, tibialis anterior (TA), and extensor digitorum longus (EDL) muscles were determined by immunofluorescence. There was no difference in the amount of Type 1 fibers for any of the muscle types examined (FIGS. 21A-21E).

Gastrocnemius, TA and EDL muscle had significant increase in Type 2a/b fibers in Fsp27^{-/-} mice compared to WT and Fsp27^{+/-} mice (FIGS. 21C-21E).

Fsp27^{-/-} mice demonstrated a significant increase in Type 2a fibers and decrease in Type 2b fibers in EDL as compared to WT (FIG. 21E). It is noted that these mice possess higher oxidative metabolism compared to WT mice. Type 2a muscle fibers are associated with higher endurance and less fatigability. Although Fsp27^{-/-} mice possess an increased amount of oxidative Type 2a fibers in their EDL, this did not result in an improvement to running endurance.

### Fsp27 Deletion Alters Lipid Profile Without Impacting Liver and Skeletal Muscle Glycogen Content

A significant decline in muscle endurance and strength in Fsp27^{-/-} mice was unexpected. To understand the underlying cause, serum lipid profile and glycogen content in the liver and the muscle of WT and Fsp27^{-/-} mice were studied.

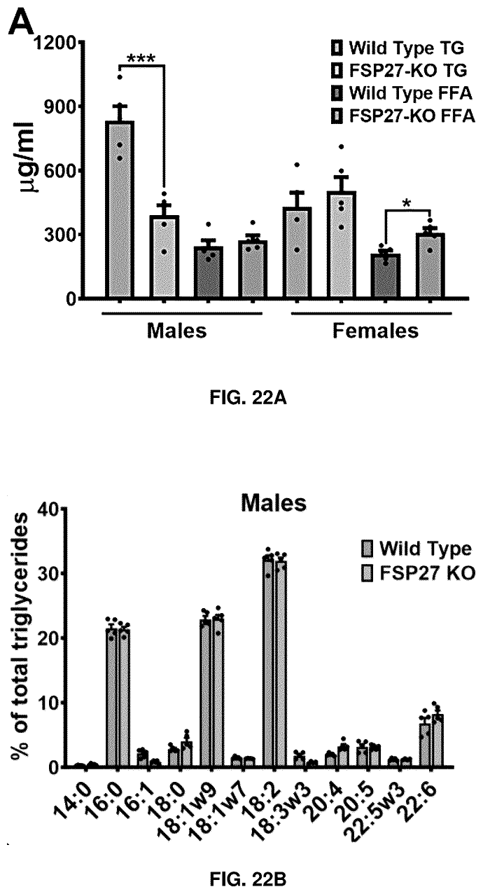

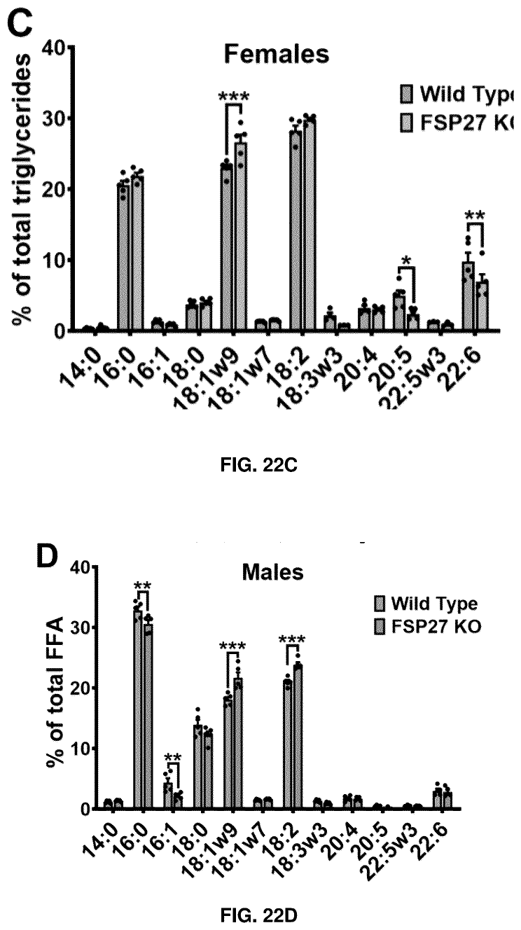

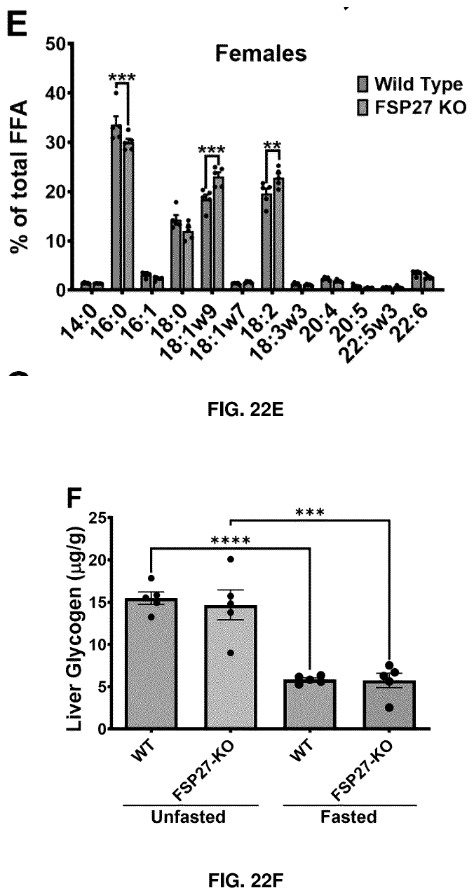

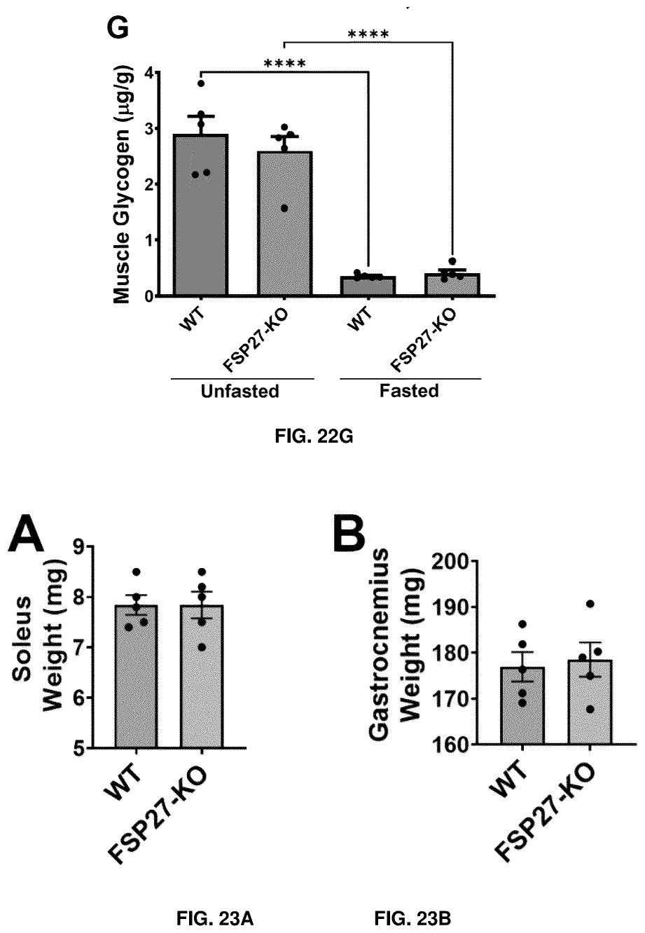

Male Fsp27^{-/-} mice showed a significant reduction in circulating TGs as compared to WT mice (FIG. 22A), while the amount of FFAs remained unchanged (FIG. 22A).

Conversely, female Fsp27^{-/-} mice had similar levels of circulating TGs but higher circulating FFAs compared to the WT (FIG. 22A). Closer examination of fatty acid species revealed that although WT males had increased circulating TGs, the percentages of each lipid species remained stable (FIGS. 22A and 22B).

In females, while the total circulating TGs were similar, composition analysis revealed differences in unsaturated fatty acids: an increase in oleic acid (18:1w9, an omega-9 fatty acid) but a decrease in eicosapentaenoic acid (20:5) and docosahexaenoic acid (22:6), both of which are omega-3 fatty acids (FIG. 22C).

Male and female mice showed changes in FFA composition which were largely similar between the sexes—a reduction in palmitic acid (16:0; saturated) in both male and female mice, but a reduction in palmitoleic acid (16:1; unsaturated) only in males as well as a consistent increase to oleic and linoleic acid for both sexes (18:2, an omega-6 fatty acid; FIGS. 22D and 22E). Glycogen stores in the liver and skeletal muscles were not affected under fed or fasted conditions in Fsp27^{-/-} animals (FIGS. 22F-22G). These data show that glycogen stores are not the cause for loss of muscle performance in Fsp27^{-/-} mice.

### Fsp27^{-/-} Mice Display Impaired Hepatic and Mitochondrial Function

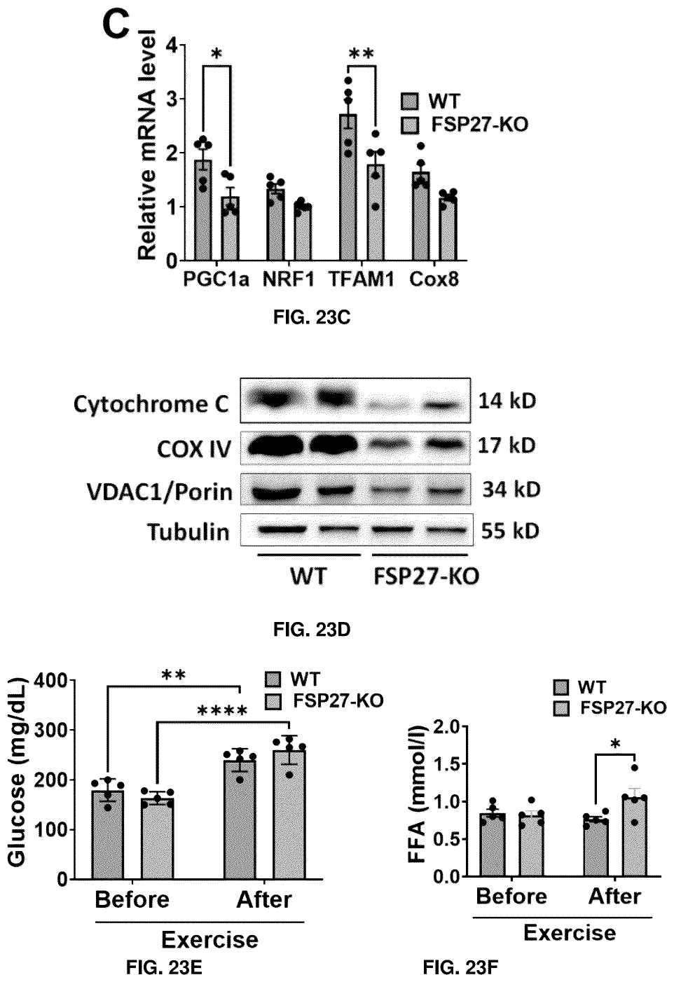

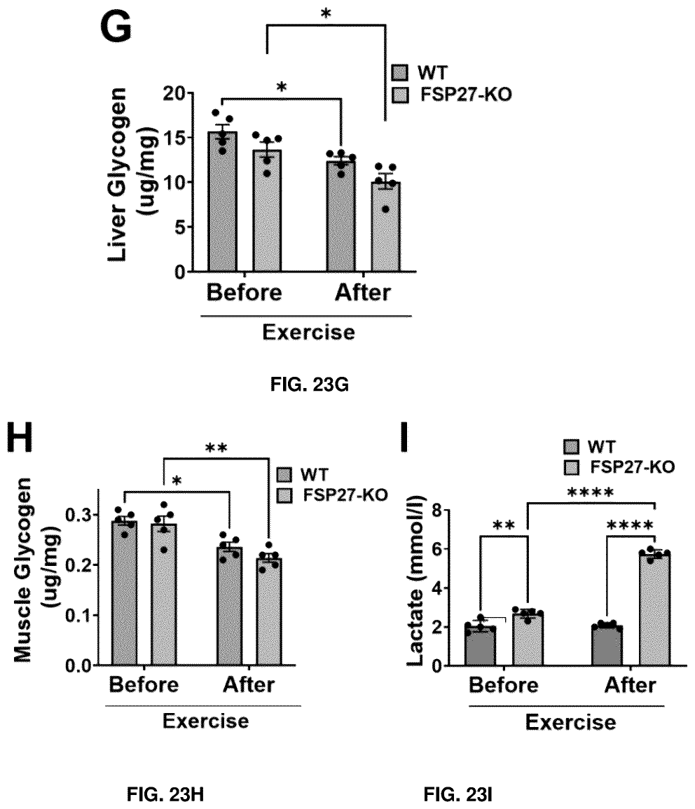

The muscle mass in these mice was measured. Soleus or gastrocnemius muscle mass was similar in WT and Fsp27^{-/-} mice (FIGS. 23A-23B), ruling out the possibility of differences in muscle mass for the subpar performance of Fsp27^{-/-} mice. Alternatively, we tested if reduced number of mitochondria in Fsp27^{-/-} mice could have caused low energy production. Interestingly, skeletal muscle of Fsp27^{-/-} mice showed significantly reduced level of Transcription Factor A, Mitochondrial (TFAM) and Peroxisome proliferator-activated receptor gamma coactivator 1-alpha (PGC1-αgene expression) (FIG. 23C). TFAM and PGC1-a regulate mitochondrial transcription and mitochondrial biogenesis, respectively.

Similarly, Nuclear Respiratory Factor 1 (NRF1) and Cytochrome c oxidase subunit 8(COX8), that regulate mitochondrial DNA replication and electron transport chain/oxidative phosphorylation, respectively, also showed reduced expression in Fsp27^{-/-} mice. Although, the reduction in NRF1 and COX8 was not significant (FIG. 23C).

We also found reduced mitochondrial density, measured by the protein expression of cytochrome C, cytochrome c oxidase IV (COX IV), and Voltage Dependent Anion Selective Channel 1 (VDAC1; aka Porin) in the muscle of Fsp27^{-/-} mice (FIG. 23D).

Next, we investigated if sufficient glucose as a fuel was in circulation in Fsp27^{-/-} mice to meet energy demands of their exercising muscles. As shown in FIG. 23E, both WT and Fsp27^{-/-} mice had comparable levels of blood glucose before or after exercise. Blood glucose levels were significantly elevated after exercise in both groups, suggestive of effective hepatic glycogenolysis (FIG. 23E). No significant change was observed in the expression of genes involved in IMTG formation (DGAT1 and DGAT2) and lipolysis (HSL and ATGL) also show a modest change.

Next, we assessed circulating FFA levels in these animals. As shown in FIG. 23F, both groups had similar FFA profile before exercise. Fsp27^{-/-} animals had significantly elevated serum FFA levels after exercise showing elevated adrenergic response or impaired mitochondrial oxidation. The latter is consistent with Fsp27^{-/-} mice having slightly reduced mitochondrial number compared to WT animals (FIGS. 23C-23D).

Glycogen levels in liver and muscles before and after exercise were examined As shown in FIGS. 23G-23H, both WT and Fsp27^{-/-} mice had similar levels of glycogen before and after exercise in liver as well as in muscles; however, glycogen levels dropped significantly in both organs in both groups of mice after exercise, showing that shortage of fuel supply could not account for poor muscle performance in Fsp27^{-/-} mice. Also, these results rule out the incapacity to mobilize fat and glycogen reserves during running. Finally, we tested circulating lactate levels in WT and Fsp27/animals. Interestingly, high levels of circulating lactate were present before exercise in Fsp27^{-/-} animals which were compounded further after exercise (FIG. 231).

### Fsp27 Deletion Alters Fat Content In The Muscle

Fsp27 is a fat-associated protein that plays an important role in fat accumulation by regulating lipid droplet accumulation and enlargement. It is now shown herein that Fsp27 is responsible for fat accumulation as a fuel source in the muscle. We performed immunostaining of fat pads and gastrocnemius muscle of wild type, Fsp27^{+/-} and Fsp27^{-/-} mice for Fsp27 using Fsp27-antibodies and Bodipy, respectively. Fsp27--mice showed negligible amounts of fat (FIGS. 23A and 23B). Interestingly, the Fsp27^{+/-} mice showed a significantly lower amount of Fsp27 and fat in the gastrocnemius muscle (FIGS. 23B and 23C), showing that the severely compromised muscle performance in Fsp27^{-/-} mice might be, at least partially, due to the lack of fat storage in muscle.

### Localization of Fat and Fsp27 in Muscle.

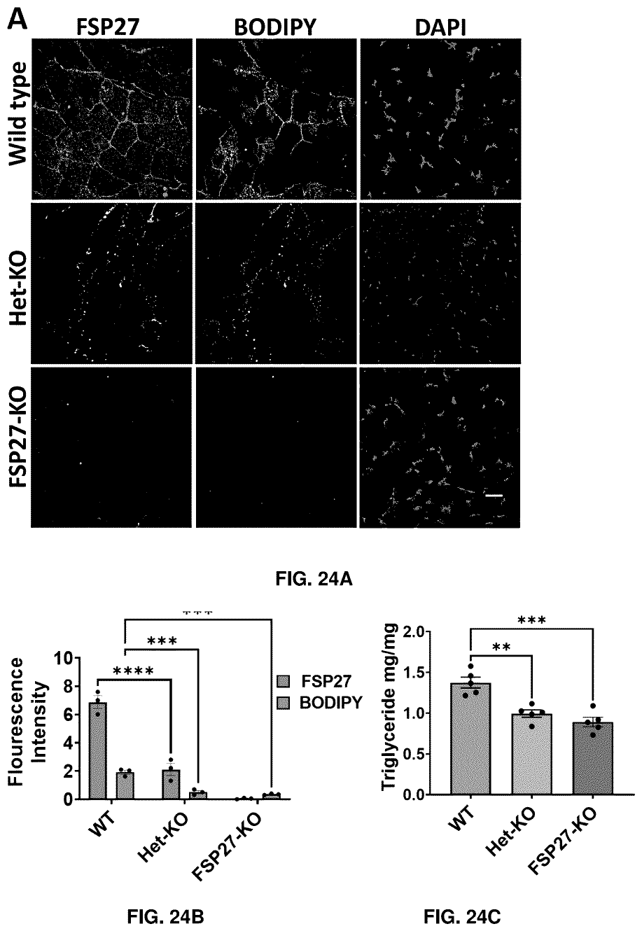

As shown in FIG. 24A, confocal microscopy images representing the localization of Fsp27 (magenta; Cy5), neutral lipid (green; BODIPY), and nuclei (blue; DAPI) in the gastrocnemius of wild type, Fsp27^{+/-} and Fsp27^{-/-} mice. FIG. 24B shows the qualitative measurement of fat staining in the gastrocnemius of wild type, Fsp27^{+/-} and Fsp27/-mice. FIG. 24C shows the Intramuscular triglyceride contents. n=5 male mice per group. Data are expressed as means ±S.E.M. For statistics one-way ANOVA followed by Tukey's multiple comparison test was performed * p<0.05,** p<0.01 *** p<0.001, **** p<0.0001.

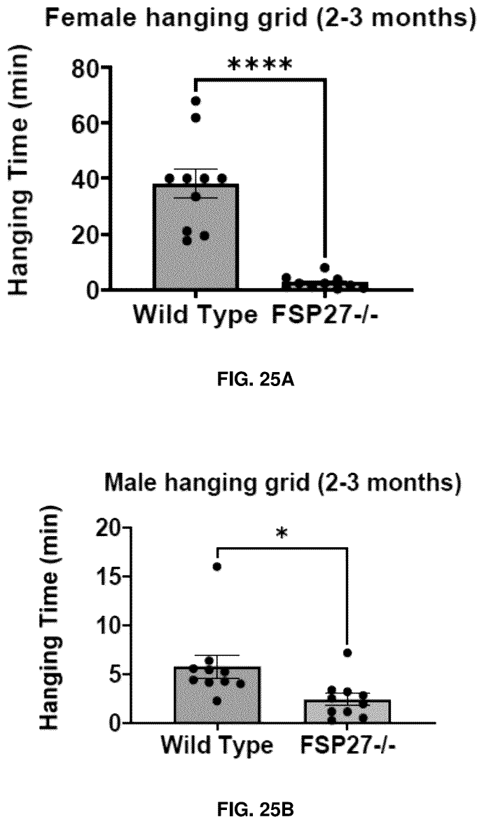

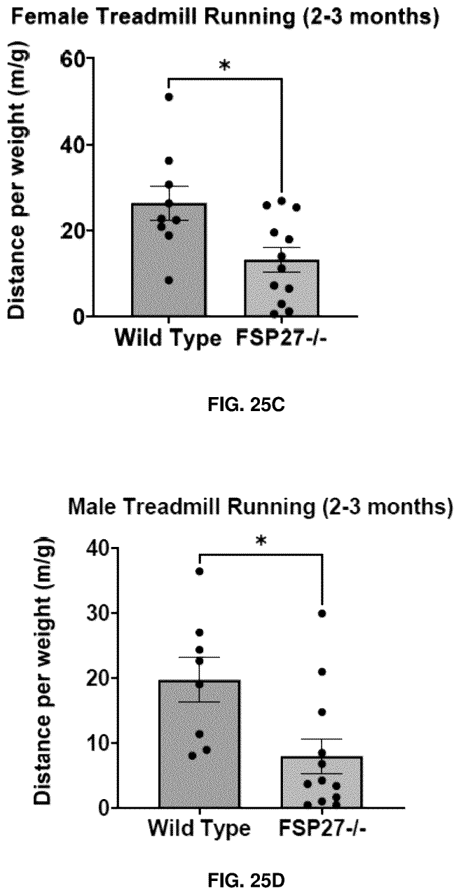

As shown in FIGS. 25A-25C: FSP27-7. mice showed reduces muscle performance in at 2-3 months of age. Four-limb hanging grid test performed on female mice (FIG. 25A), male mice (FIG. 25B). For treadmill running endurance test mice was run until exhaustion Male mice (FIG. 25C) female mice (FIG. 25D) (n>8 mice per group).

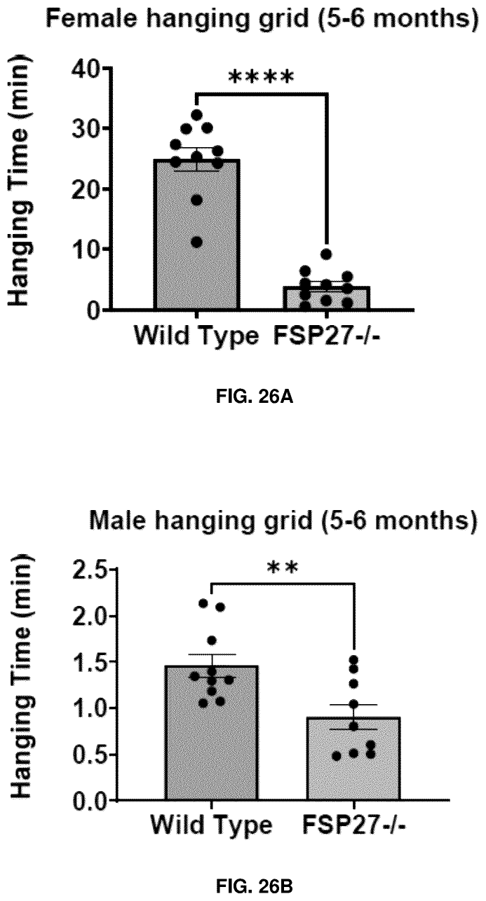

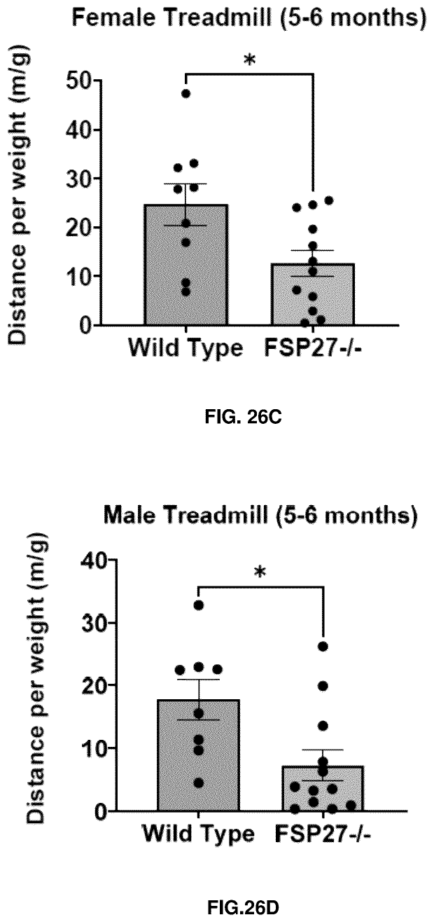

As shown in FIGS. 26A-26D: FSP27-1. mice showed reduces muscle performance in at 5-6 months of age. Four-limb hanging grid test performed on female mice (FIG. 26A), male mice (FIG. 26B). For treadmill running endurance test mice was run until exhaustion female mice (FIG. 26C) male mice (D) (n≥8 mice per group).

Fsp27 plays a role in muscular-fat storage, muscle endurance, and muscle strength. Those studies were performed in a cohorts of 8-10 months age and the result demonstrate reduced fat storage, muscle endurance and strength in Fsp27--mice compared to wild type mice.

Both 2-3-months and 5-6-months old Fsp27^{-/-} mice exhibited significant decline hanging endurance compared to wild type mice (FIG. 25A-25B, and FIG. 26A-26B). The decline in hanging endurance time was predominant in female mice compared to male mice. In treadmill running test both male and female Fsp27^{-/-} mice fatigued sooner and ran at least 50% less distance compared to wildtype mice. The trend was similar in both 2-3 and 5-6 months age group (FIG. 25C-FIG. 25D and FIG. 26C-FIG. 26D).

### DISCUSSION

Example 2 shows a previously unidentified role of Fsp27 in muscle performance. Fsp27^{-/-} mice display improved insulin sensitivity, and glucose tolerance compared to WT and Fsp27^{-/-} + mice. We found a shift towards hybrid type 2a/b muscle fibers in Fsp27^{-/-} mice, showing that Fsp27^{-/-} mice would have higher muscle endurance.

Surprisingly, Fsp27-mice appeared to be less mobile during the day-night transition. Interestingly, Fsp27^{+/-} and Fsp27^{-/-} mice displayed significant differences in endurance at both physiological challenges compared to the WT mice. The loss of exercise capacity was dramatic, with both male and female Fsp27^{-/-} mice becoming exhausted at approximately 15% of the distance covered by wild-type controls. Similarly, Fsp27^{-/-} mice were unable to perform comparably to controls on the four-limb hanging grid test, with a holding impulse that measured 30% of what was attainable by WT mice. It was initially believed that reduced energy reserves or impaired capacity to mobilize fat and glycogen reserves might contribute to the poor performance in physiological challenge; however, this data showed that liver and muscle glycogen contents remained unchanged before and after exercise while serum glucose and serum FFA levels were elevated in Fsp27^{-/-} mice. The heterozygous Fsp27^{+/-} mice underperformed in exercise, endurance, and strength tasks compared to WT mice, showing that Fsp27 haploinsufficiency compromises muscle performance. Sexual dimorphism was not observed. Overall, besides having higher glucose tolerance, Fsp27^{-/-} caused marked deficiencies in fat storage and muscle performance.

The two main fuels for muscle metabolism are carbohydrates and fat, and fat is important as a support nutrient for energy during exercise. At low-to-moderate intensities of exercise, the primary fuel sources for skeletal muscle are glucose, derived from liver glycogen, and FFAs, primarily derived from adipose tissue lipolysis. During long-duration exercise, fatty acids are oxidized to be used as fuel for the muscle. A large portion of available fatty acids are stored in adipose tissue as TAGs, but a modest amount remains stored in the muscle itself and within blood plasma. Fsp27^{-/-} mice are intriguing because they retain TAGs and FFAs within their blood plasma (FIG. 22A), but their ability to store fats within WAT (FIGS. 17D, 17E) and muscle is severely compromised (FIG. 22H). Their available fatty acids stores, therefore, come from the blood plasma only, which is minuscule in comparison to adipose and muscle. Despite having improved metabolic parameters and fatty acid oxidation rates, Fsp27^{-/-} mice were unable to perform as well, as WT or Fsp27^{+/-} mice on the running endurance and hanging grid challenge. It is now believed that the lack of fat stores in the WAT and muscle of Fsp27^{-/-} mice caused the observed decline in running endurance. The amount of intramuscular fat in Fsp27^{-/-} mice was lower than the WT mice but higher than Fsp27^{-/-} mice, which related to their endurance capacity.

Example 2 shows that Fsp27 is expressed in muscles and plays an important role in fat storage in the muscle, as it does in adipocytes. It is now believed that Fsp27 regulates lipid droplet dynamics to store triglycerides in the muscles, and regulates their breakdown via lipolysis, in a similar manner to its function in adipocytes. Although glucose levels before and after exercise were similar in WT and Fsp27^{-/-} mice, FFAs were higher in Fsp27^{-/-} mice after the exercise.

Example 2 also shows that the insulin-sensitizing effects observed in young, male mice lacking Fsp27 are extended to adult (8-10 months old) mice of both sexes. However, the enhanced absorption of glucose was the primary retained metabolic factor in these experiments, as we did not observe any significant differences during the insulin challenge. This is likely an age-related effect as the previous work was performed on a much younger set of mice that were highly metabolically active. Interestingly, amongst both male and female mice, we did not detect a difference in the fasting blood glucose of the three genotypes. This shows that the enhanced glucose metabolism seen in Fsp27^{-/-} mice is the result of a necessity, as the increased systemic fatty acids are constantly being oxidized even at rest.

Fsp27 has a role in lipid metabolism within the adipose tissue and liver. Example 2 now shows a previously unidentified role of FSP27 in muscle performance. In addition, Example 2identifies a metabolic paradox in which Fsp27-KO mice presumed to be metabolically healthy based on glucose utilization and insulin sensitivity are, in fact, unhealthy in terms of exercise capacity and muscular performance.

Example 2 shows that directly administering to muscle cells and tissues of the subject can increase muscle function in subjects in need thereof. In certain embodiments, a recombinant peptide comprising a fat specific protein 27 (FSP27) is delivered in an amount sufficient to increase muscle in muscle cells and tissue without the need for gene delivery mechanisms.

### Pharmaceutical Compositions

A pharmaceutical composition as described herein may be formulated with any pharmaceutically acceptable excipients, diluents, or carriers. A composition disclosed herein may comprise different types of carriers depending on whether it is to be administered in solid, liquid, or aerosol form, and whether it needs to be sterile for such routes of administration as injection. Compositions disclosed herein can be administered in a suitable manner, including, but not limited to topically (i.e., transdermal), subcutaneously, by localized perfusion bathing target cells directly, via a lavage, in creams, in lipid compositions (e.g., liposomes), formulated as elixirs or solutions for convenient topical administration, formulated as sustained release dosage forms, or by other method or any combination of the forgoing as would be known to one of ordinary skill in the art (see, for example, Remington's Pharmaceutical Sciences, 2003, incorporated herein by reference).

The compositions provided herein are useful for treating animals, such as humans. A method of treating a human patient according to the present disclosure includes the administration of a composition, as described herein.

The phrases “pharmaceutical” or “pharmacologically acceptable” refer to molecular entities and compositions that produce no adverse, allergic, or other untoward reaction when administered to an animal, such as, for example, a human. A carrier or diluent may be a solid, semi-solid, or liquid material which serves as a vehicle, excipient, or medium for the active therapeutic substance. Some examples of the diluents or carriers which may be employed in the pharmaceutical compositions of the present disclosure are lactose, dextrose, sucrose, sorbitol, mannitol, propylene glycol, liquid paraffin, white soft paraffin, kaolin, fumed silicon dioxide, microcrystalline cellulose, calcium silicate, silica, polyvinylpyrrolidone, cetostearyl alcohol, starch, modified starches, gum acacia, calcium phosphate, cocoa butter, ethoxylated esters, oil of theobroma, arachis oil, alginates, tragacanth, gelatin, syrup, methyl cellulose, polyoxyethylene sorbitan monolaurate, ethyl lactate, methyl and propyl hydroxybenzoate, sorbitan trioleate, sorbitan sesquioleate and oleyl alcohol, and propellants such as trichloromonofluoromethane, dichlorodifluoromethane, and dichlorotetrafluoroethane.

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols, and mixtures thereof, and in oils. Under ordinary conditions of storage and use, these preparations contain a preservative to prevent the growth of microorganisms. The pharmaceutical forms suitable for use include sterile aqueous solutions or dispersions and sterile powders for the extemporaneous preparation of sterile solutions or dispersions. In certain cases, the form should be sterile and should be fluid to the extent that easy injectability exists. It should be stable under the conditions of manufacture and storage and may optionally be preserved against the contaminating action of microorganisms, such as bacteria and fungi. The carrier can be a solvent or dispersion medium containing, for example, water, ethanol, a polyol (i.e., glycerol, propylene glycol, and liquid polyethylene glycol, and the like), suitable mixtures thereof, and/or vegetable oils. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, such as, but not limited to, sugars or sodium chloride.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream, and petrolatum as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

It is further envisioned the compositions disclosed herein may be delivered via an aerosol.

The term aerosol refers to a colloidal system of finely divided solid or liquid particles dispersed in a liquefied or pressurized gas propellant. The typical aerosol comprises a suspension of active ingredients in liquid propellant or a mixture of liquid propellant and a suitable solvent. Suitable propellants include hydrocarbons and hydrocarbon ethers. Suitable containers can vary according to the pressure requirements of the propellant. Administration of the aerosol can vary according to subject's age, weight, and the severity and response of the symptoms.

### Dosage

The actual dosage amount of a composition disclosed herein administered to an animal or human patient can be determined by physical and physiological factors such as body weight, severity of condition, the type of disease being treated, previous or concurrent therapeutic interventions, idiopathy of the patient, and the route of administration. Depending upon the dosage and the route of administration, the number of administrations of a preferred dosage and/or an effective amount may vary according to the response of the subject. The compounds of the present disclosure are generally effective over a wide dosage range. The practitioner responsible for administration can, in any event, determine the concentration of active ingredient(s) in a composition and appropriate dose(s) for the individual subject.

Naturally, the amount of active compound(s) in each therapeutically useful composition may be prepared in such a way that a suitable dosage can be obtained in any given unit dose of the compound. Factors such as solubility, bioavailability, biological half-life, route of administration, product shelf life, as well as other pharmacological considerations can be contemplated by those preparing such pharmaceutical formulations, and as such, a variety of dosages and treatment regimens may be desirable.

In other non-limiting examples, a dose may also comprise from about 1

microgram/kg/body weight, about 5 microgram/kg/body weight, about 10 microgram/kg/body weight, about 50 microgram/kg/body weight, about 100 microgram/kg/body weight, about 200microgram/kg/body weight, about 350 microgram/kg/body weight, about 500 microgram/kg/body weight, about 1 milligram/kg/body weight, about 5 milligram/kg/body weight, about 10milligram/kg/body weight, about 50 milligram/kg/body weight, about 100 milligram/kg/body weight, about 200 milligram/kg/body weight, about 350 milligram/kg/body weight, about 500milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5microgram/kg/body weight to about 500 milligram/kg/body weight, etc., can be administered, based on the numbers described above. The dosages can depend on many factors, and can in any event be determined by a suitable practitioner. Therefore, the dosages described herein are not intended to be limiting

In some embodiments, the compositions further include an additional active ingredient.

The preparation of a pharmaceutical composition that contains at least one compound or additional active ingredient can be known to those of skill in the art in light of the present disclosure, as exemplified by Remington's Pharmaceutical Sciences, 2003, incorporated herein by reference. Moreover, for animal (e.g., human) administration, it can be understood that preparations should meet sterility, pyrogenicity, and general safety and purity standards as required by the FDA Office of Biological Standards.

Packaging of the Composition

After formulation, the composition is packaged in a manner suitable for delivery and use by an end user. In one embodiment, the composition is placed into an appropriate dispenser and shipped to the end user. Examples of final container may include a pump bottle, squeeze bottle, jar, tube, capsule or vial.

The compositions and methods described herein can be embodied as parts of a kit or kits. A non-limiting example of such a kit comprises the ingredients for preparing a composition, where the containers may or may not be present in a combined configuration. In certain embodiments, the kits further comprise a means for administering the composition, such as a topical applicator, or a syringe. The kits may further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive, CD-ROM, or diskette. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

what is claimed is:

1. A method for improving muscle performance in a subject in need thereof, comprising:
directly administering to muscle cells and tissues, without the need for gene delivery mechanisms to a subject in need thereof, a recombinant peptide comprising a fat specific protein 27 (FSP27), in an amount sufficient to increase muscle function in the muscle cells and tissue.

2. The method according to claim 1, wherein the recombinant peptide comprises at least one of: SEQ ID NOs: 3, 4, 5, 6, 7, or the 120-220 aa of SEQ ID NO: 2.

3. The method of claim 1, wherein the subject is a human.

4. The method of claim 1, wherein the composition is administered to the subject parenterally.

5. The method according to claim 1, wherein muscle performance includes higher endurance and less fatigability, than is a subject not receiving the FSP27 medicament.

6. A method for regulating lipid droplet dynamics as a way to store triglycerides in muscles in a subject in need thereof, and regulating lipid droplet breakdown via lipolysis in the subject in need thereof, comprising:
administering to the subject an FSP27 medicament or a pharmaceutically acceptable composition thereof.

7. The method according to claim 6, the FSP27 medicament is administered to muscle tissue.

8. A method for reducing the amount of fat in muscle tissue in a subject in need thereof, comprising:
administering to the subject an effective amount of an FSP27 medicament to a subject in need thereof.

9. A pharmaceutical composition comprising one or more FSP27 medicaments, or one or more pharmaceutically acceptable modifications of FSP27 thereof, optionally together with one or more inert carriers and/or diluents, the FSP27 medicament being present in an amount sufficient to treat increase muscle performance.


SEQUENCE LISTING (7 sequences)

SEQ ID NO: 1 - DNA, 11 bp, unidentified
  source 1..11: mol_type = unassigned DNA; note = Description of Unknown: Egr1 binding site
ccgccctcacc

SEQ ID NO: 2 - protein, 238 aa, Homo sapiens
MEYAMKSLSLLYPKSLSRHVSVRTSVVTQQLLSEPSPKAPRARPCRVSTADRSVRKGIMAYSLEDLLLKVRDTLMLADKPFFLVLEEDGTTVETEEYFQALAGDTVFMVLQKGQKWQPPSEQGTRHPLSLSHKPAKKIDVARVTFDLYKLNPQDFIGCLNVKATFYDTYSLSYDLHCCGAKRIMKEAFRWALFSMQATGHVLLGTSCYLQQLLDATEEGQPPKGKASSLIPTCLKILQ

SEQ ID NO: 3 - protein, 239 aa, Mus sp.
MDYAMKSLSLLYPRSLSRHVAVSTAVVTQQLVSKPSRETPRARPCRVSTADRKVRKGIMAHSLEDLLNKVQDILKLKDKPFSLVLEEDGTIVETEEYFQALAKDTMFMVLLKGQKWKPPSEQRKKRAQLALSQKPTKKIDVARVTFDLYKLNPQDFIGCLNVKATLYDTYSLSYDLHCYKAKRIVKEMLRWTLFSMQATGHMLLGTSSYMQQFLDATEEEQPAKAKPSSLLPACLKMLQ

SEQ ID NO: 4 - protein, 246 aa, Macaca mulatta
MGSLRKGFGLLKDPWERAGFDWAACLRVSVRTSVVTQQLLSEPSPEAPRARPCRVSTADRSVRKGIMAYSLEDLLLKVRDTLMLADKPFFLVLEEDGTTVETEEYFQALAGDIVFMVLQKGQKWQPPSEQGTRHPQSLSHKPAKKIDVARVTFDLYKLNPQDFIGCLNVKATFYDTYSLSYDLHCCGAKRIVKEALRWALFSMQATGHVMLGTSCYLQQLLDATEEGQPPKGKASSLIPTCLRILQ

SEQ ID NO: 5 - protein, 238 aa, Canis lupus
  source 1..238: note = familiaris
MEYAMKSLSLLYPKSLSRHVAVSTSVVTQQLSSKSSLEAPKARPCRVSTADRSVRKGIIAHSLKDLLNKVRDTLLLADKPFYLVLEEDGTTVETEEYFQALADDTVFMVLQKGQKWQPPQEQGSRYQLSLSHKPAKKIDVAQVTFDLYKMNPQDFIGCLNVKATLYGTYSLSYDLHCYGAKRIMKEVLRWALFSMKTTGHVLLGTSCYMQQLLDATEGGQPPEGKARSLIPTSLKMLQ

SEQ ID NO: 6 - protein, 222 aa, Bos taurus
MAMYTAVSTSVVTQQQLSEPSAEAPRARPCRVTTADRSVRKGIMVHSLEDLHVKVQDTLMLAYRPFFLVLEEDGTTVETEEYFQSLADDTVFMVLHKGQKWQPPSEQSTRYQLALSHKPAKIDVAQVTFDLYKVNPQDFIGCLNVKATLYGTYSVSYDLHCSGAKRIMKEALRWALFSMRTTGHMLLGTSCYLQQLLDATEREQPPKSKAASLIPTSLKMLQ

SEQ ID NO: 7 - protein, 236 aa, Xenopus sp.
MEYAMKSLSLLSPKSLTKCVSVSASMTQQLLSRPVSKPRPFRVCNSNRSLRKGIVANSLEDLINKTQDALLMLEAITLVLDEDGTCVDTEEFFRSLDDGAVFMALAKGQKWKPTENSGYHLSLTKKPARKIDVACVSFDLYKNHPRDFIGCLNVKATLYGTYSLSYDLQCYGAKRMVKEALRWTLYTMQATGHVLLGTSCYMKQLLDATERPVTEEEKSSTTLRDFIPFSPWKMLQ